SEQUENCE LISTING (6 sequences)

SEQ ID NO: 1 - protein, 44 aa, Pediococcus acidilactici MM33
KYYGNGVTCGKHSCSVDWGKATTCIINNGAMAWATGGHQGNHKC

SEQ ID NO: 2 - DNA, 34 bp, Artificial Sequence
  Synthetic primer
aaagaattcatgaaaaaaattgaaaaattaactg

SEQ ID NO: 3 - DNA, 36 bp, Artificial Sequence
  Synthetic primer
aaaggtaccctagcatttatgattaccttgatgtcc

SEQ ID NO: 4 - DNA, 29 bp, Artificial Sequence
  Synthetic primer
ccggaattcataaggaggcactcaaaatg

SEQ ID NO: 5 - DNA, 29 bp, Artificial Sequence
  Synthetic primer
cggggtacctactatcctttgatttggtt

SEQ ID NO: 6 - DNA, 200 bp, Lactococcus lactis MM19
  misc_feature (2)..(2): n is a, c, g, or t
tncnaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataaccaaatcaaaggatagtaggtaccccgagana

(19) United States
(12) Patent Application Publication
(10) Pub. No.: US 2011/0236359 A1
(43) Pub. Date: Sep. 29, 2011

(54) Antimicrobial Activity of Bacteriocin-Producing Lactic Acid Bacteria

(75) Inventors: Monique Lacroix, Saint-Lambert (CA); Mathieu Millette, Delson (CA)

(73) Assignee: INSTITUT NATIONAL DE LA RECHERCHE SCIENTIFIQUE, Quebec, QC (CA)

(21) Appl. No.: 12/744,280

(22) PCT Filed: Sep. 4, 2008
(86) PCT No.: PCT/CA08/01576

§ 371 Date: May 24, 2011

Related U.S. Application Data
(60) Provisional application No. 60/935,892, filed on Sep. 5, 2007.

(51) Int. Cl. A01N 63/00; C12N 1/20; C07K 14/195; A61K 35/74; A61K 38/16; A01N 37/18; A01P 1/00; A61P 31/04; A23L 1/30; A23L 3/34

(52) U.S. Cl. 424/93.45; 435/252.9; 530/324; 435/252.1; 424/93.4; 514/2.4; 514/2.7; 426/61; 426/2

(57) ABSTRACT

The present invention relates to novel bacteriocin-producing lactic acid bacteria strains Lactococcus lactis subsp. lactis MM19 accession number NML-080508-01 and Pediococcus acidilactici MM33 accession number NML-080508-02 isolated from the human gut. The strains L. lactis subsp. lactis MM19 and P. acidilactici MM33 and the bacteriocins produced by these strains are useful for inhibiting microbial growth in food products, and for inhibiting microbial infection or colonization of a mammal.

### FIELD OF INVENTION

This invention relates to the antimicrobial activity of novel bacteriocin-producing lactic acid bacteria.

### BACKGROUND OF THE INVENTION

Lactic acid bacteria (LAB) are Gram-positive bacteria that produce lactic acid by glucose fermentation. Production of lactic acid by LAB may prevent the growth of spoilage organisms and some LAB may be used to extend the shelf life of food. Several strains of LAB are recognized as safe and may be used in the production of cheese, fermented sausage and other fermented food. Production of lactic acid by LAB may also prevent the growth of pathogenic bacteria.

Probiotic bacteria are microorganisms that can alter the intestinal microbiota and exert beneficial health effect for the host. Maintenance of a healthy intestinal microbiota is important in order to protect human health and probiotic LAB are known to contribute to this state. Some mechanisms of action have been proposed to explain the efficacy of these probiotics. The proposed mechanisms include, for example, production of antagonistic compounds including antimicrobial substances, competition for mucosal surfaces as well as for available nutrients, immunomodulation, promotion of lactose digestion, etc.

Exemplary antimicrobial compounds are bacteriocins, ribosomally synthesized bactericidal peptides that are produced by some microorganisms in all major lineages of Eubacteria and Archaebacteria (Riley and Gordon, 1999). Bacteriocins from LAB are low molecular weight, cationic, amphiphilic molecules (Drider et al. 2006). Exemplary bacteriocins produced by LAB may include various nisins, lacticins, lactostrepcins, lactococcins, lactocins, pediocins, etc. The relationship between probiotic characteristics and bacteriocin-producing capacity is poorly understood.

There is a continual need for natural microbial growth control agents and natural food preservatives bearing new and improved properties; the present invention seeks to meet these and other needs.

The present description refers to a number of documents, the content of which is herein incorporated by reference in their entirety.

### SUMMARY OF THE INVENTION

In the present invention, novel bacteriocin-producing lactic acid bacteria strains of L. lactis subsp. lactis and P. acidilactici were isolated from the human gut.

In a first aspect thereof, the present invention relates to isolated lactic acid bacteria that may have the identifying characteristics of strain Lactococcus lactis subsp. lactis MM19 accession number NML-080508-01 and/or Pediococcus acidilactici MM33 accession number NML-080508-02. A (pure) culture of such isolated lactic acid bacteria, a cell-free culture supernatant of such (pure) culture and/or an isolated bacteriocin produced by such lactic acid bacteria are also within the scope of the present invention.

In another aspect thereof, the present invention relates to a composition that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and a carrier.

In an additional aspect thereof, the present invention relates to a food product that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof.

In a further aspect thereof, the present invention relates to a method for inhibiting microbial growth. The method may comprise the step of contacting a microbe with the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention, the food product that may comprise the isolated lactic acid bacteria strains of the present invention and/or combination thereof.

In yet a further aspect thereof, the present invention relates to a method for modulating the gut flora in a mammal in need thereof. The method may comprise the step of administering an effective amount of a composition that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof. The method may comprise the step of administering a food product that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof.

In an additional aspect thereof, the present invention relates to a method for preventing and/or treating a microbial infection in a mammal in need thereof. The method may comprise administering an effective amount of a composition that may comprise the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and a carrier to a mammal in need thereof. The method may also comprise administering a food product that may comprise the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof.

In yet an additional aspect thereof, the present invention relates to a method for preventing and/or reducing the level of microbial colonization in a food product. The method may comprise contacting the food product with an effective amount of a composition that may comprise the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof.

In a further aspect thereof, the present invention relates to a kit. The kit may comprise at least one container that may contain the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention, the food product that may comprise the isolated lactic acid bacteria strain of the present invention and/or combination thereof.

Further scope, applicability and advantages of the present invention will become apparent from the non-restrictive detailed description given hereinafter. It should be understood, however, that this detailed description, while indicating exemplary embodiments of the invention, is given by way of example only, with reference to the accompanying drawings.

### BRIEF DESCRIPTION OF THE DRAWINGS

In drawings which illustrates non-limitative exemplary embodiments of the present disclosure,

FIG. 1 shows the effects of exposure to various temperature for 15 minutes (♦), 30 minutes (□) or 60 minutes (▴) on the antimicrobial activity of cell-free supernatants from cultures of: (a) Lactococcus lactis subsp. lactis MM19 or (b) Pediococcus acidilactici MM33. Results are means of three individual assays with a standard deviation (SD) less than 5% about the mean.

FIG. 2 shows the effects of pH on the relative antimicrobial activities of cell-free supernatants from cultures of Lactococcus lactis subsp. lactis MM19 (□) or Pediococcus acidilactici MM33 (▴). Results are means of three individual assays with a standard deviation less than 5% about the mean.

FIG. 3 shows the number of bacteria (∘), amounts of bacteriocin (▪) and pH values (▴) in cultures of: (a) Lactococcus lactis subsp. lactis MM19 or (b) Pediococcus acidilactici MM33 in lactobacilli MRS broth during growth at 36±1° C. under aerobic conditions. Results are means of three individual assays with a standard deviation less than 5% about the mean except for the number of bacteria.

FIG. 4 shows the SDS-PAGE profiles of the bacteriocins formed in the supernatants of cultures of Lactococcus lactis subsp. lactis MM19 and Pediococcus acidilactici MM33. Inhibition zones formed by active components are shown. Lane 1, molecular weight marker Mark 12; lane 2, bacteriocins from Lactococcus lactis subsp. lactis MM19; lane 3: bacteriocins from P. acidilactici MM33.

FIG. 5 shows a well diffusion assay of neutralized cell-free supernatant from Lactococcus lactis subsp. lactis MM19 (N) or from P. acidilactici MM33 (P) against a clinical isolate of vancomycin-resistant Enterococcus faecium in BHI agar without proteases (A) or in the presence of 15 U/ml of proteases type XIV from Streptomyces griseus (B).

FIG. 6 shows a cation-exchange chromatogram of the antimicrobial peptide produced by Pediococccus acidilactici MM33. Absorbance at 220 nm (line); % NaCl gradient (large dots), and inhibition zone diameter (mm) against Lactobacillus sakei ATCC 15521 (small dots).

FIG. 7 (A) SDS-PAGE profile of the fractions recovered along the purification steps of the pediocin secreted by Pediococcus acidilactici MM33. The black arrow indicates the band of purified pediocin. (B) Inhibition zones formed by fractions obtained during pediocin purification and tested against Lactobacillus sakei ATCC 15521. Lane M: Molecular weight marker (Mark 12; Invitrogen); lane 1: CFS from P. acidilactici MM33 (Fraction I); lane 2: Fraction IIc-pool 1; lane 3: Fraction IIc-pool 2; lane 4: Fraction V-pool 1; lane 5: Fraction V-pool 2.

FIG. 8 shows the antimicrobial activity of the cell-free supernatant produced by Pediococcus acidilactici MM33 (box A), P. acidilactici MM33A (box B), P. acidilactici MM33 in presence of proteases (box C) and P. acidilactici MM33A in presence of proteases (box D). The two wells of each square are duplicate samples.

FIG. 9 Listeria monocytogenes HPB 2812 serotype ½a growth in tryptic soy broth in presence of: 0 (♦); 100 (□); 200 (▴); 400 () and 800 (x) AU ml⁻¹ of purified pediocin produced by Pediococcus acidilactici MM33. The black arrow indicates the moment of pediocin addition.

FIG. 10 shows total culturable LAB concentration in faeces of C57BI/6 mice following a daily ingestion of Lactococcus lactis subsp. lactis MM19, P. acidilactici MM33 or P. acidilactici MM33A. An asterisk indicates a significant variation (P≦0.05) as compared to the bacterial concentration of day 1 and the same day of the PBS control. Error bars represent the standard deviation.

FIG. 11 shows total culturable Enterobacteriaceae concentration in faeces of C57BI/6 mice following a daily ingestion of Lactococcus lactis subsp. lactis MM19, P. acidilactici MM33 or P. acidilactici MM33A. An asterisk indicates a significant variation (P≦0.05) as compared to the bacterial concentration during day 1 of the experimental group and the same day of the PBS control. Error bars represent the standard deviation.

FIG. 12 shows total culturable mesophilic anaerobes concentration in faeces of C57BI/6 mice following a daily ingestion of Lactococcus lactis subsp. lactis MM19, P. acidilactici MM33 or P. acidilactici MM33A. An asterisk indicates a significant variation (P≦0.05) as compared to the bacterial concentration during day 1 of the experimental group and the same day of the PBS control. Error bars represent the standard deviation.

FIG. 13 shows changes in density of total vancomycin-resistant Enterococcus in VRE colonized CF-1 mice treated with PBS (—∘—), bacitracin (⋄), Lactococcus lactis subsp. lactis MM19 (▴), P. acidilactici MM33 (▪) and P. acidilactici MM33A (x). The intragastric VRE infection was occurred on day 0. The oral administration of bacitracin began the day after the infection and last for three days until it was discontinued and replaced by PBS feeding. The oral administration of LAB and PBS began 7 days before the infection and was discontinued the eighth day after the infection. Error bars represent the standard deviation.

### DETAILED DESCRIPTION

In order to provide a clear and consistent understanding of the terms used in the present disclosure, a number of definitions are provided below. Unless defined otherwise, all technical and scientific terms as used herein have the same meaning as commonly understood to one of ordinary skill in the art to which this invention pertains. Where a value is explicitly recited, it is to be understood that values which are about the same quantity or amount as the recited value are also within the scope of the invention as are ranges based thereon.

In a first aspect thereof, the present invention relates to an isolated lactic acid bacteria that may have the identifying characteristics of strain Lactococcus lactis subsp. lactis MM19 and/or Pediococcus acidilactici MM33 as described herein.

Samples of Lactococcus lactis subsp. lactis MM19 and/or Pediococcus acidilactici MM33 strains were deposited at the National Microbiology Laboratory (NML; 1015 Arlington Street, Winnipeg, Manitoba, Canada, R3E 3R2) under the terms of the Budapest Treaty on May 8, 2008. Lactococcus lactis subsp. lactis MM19 and Pediococcus acidilactici MM33 strains were respectively assigned accession numbers 080508-01 and 080508-02 and are herein referred to Lactococcus lactis subsp. lactis MM19 accession number NML-080508-01 and Pediococcus acidilactici accession number NML-080508-02.

The present invention also relates to a culture of an isolated lactic acid bacteria that may have the identifying characteristics of a strain selected from Lactococcus lactis subsp. lactis MM19 and/or Pediococcus acidilactici MM33. As used herein, a “culture” of isolated lactic acid bacteria refers to a population of lactic acid bacterial cells that are growing and/or have grown (have reached a stationary and/or a plateau phase) in any suitable culture media under any appropriate growth conditions as known to a person skilled in the art of bacteria culturing. A culture media encompasses any liquid, semi-solid or solid preparation that may allow the growth and/or maintenance and/or survival and/or reproduction of bacterial cells. In an exemplary embodiment of the present invention, the culture is a pure culture, that is, a culture of lactic acid bacterial cells growing and/or that has grown in the absence of any other bacterial species. In an exemplary embodiment of the present invention, a culture media may be suitable for ingestion by a mammal, such as a human being.

In exemplary embodiments of the present invention, lactic acid bacteria strains may be bile-salt resistant, gastric acid resistant and/or combination thereof. By “resistant” it is meant that in presence of a given compound, the lactic acid bacteria strains may still grow and/or remain alive (not necessarily growing). Bile salt resistance encompasses for example, resistance to bile salt concentrations of 0.1 to 12% (w/v), 0.1 to 20% (w/v), and/or bile salt concentrations above 1%, above 5%, above 11%, above 15% and/or above 19%. Acid resistance may encompass resisting to a pH lower than 3, lower than 2.5 and/or lower than 2. Lactic acid bacteria strains of the invention may also be γ-irradiation resistant, for example, they may be resistant to irradiation doses of between 0-10 KGy, 0-12 KGy, 0-15 KGy, 0-20 KGy, 0-25 KGy, 0-30 KGy, 0-35 KGy, 0-40 KGy, 0-41 KGy and/or irradiation doses more than 1 KGy, more than 2 KGy, more than 3 KGy, more than 4 KGy, more than 5 KGy, more than 6 KGy, more than 7 KGy, more than 8 KGy, more than 9 KGy, more than 10 KGy, more than 15 KGy, more than 16 KGy, more than 17 KGy, more than 20 KGy, more than 25 KGy, more than 30 KGy, more than 35 KGy and/or more than 40 KGy. It is to be understood that any specified range or group is as a shorthand way of referring to each and every member of a range or group individually as well as each and every possible sub-ranges or sub-groups encompassed therein and similarly with respect to any sub-ranges or sub-groups therein. The present invention relates to and explicitly incorporates each and every specific member and combination of sub-ranges or sub-groups therein whatsoever. Thus, for example, when it is said that a bile salt resistance may be between 0.1 to 20% (w/v), the bile salt concentration may be 0.5% (w/v), 1% (w/v), 3% (w/v), 5% (w/v), 7% (w/v), 9% (w/v), 11% (w/v), 12% (w/v), 13% (w/v), 15% (w/v), 17% (w/v), 19% (w/v), 20% (w/v) and/or any value therebetween. As another example, when it is said that irradiation doses resistance may be between 0-10 KGy, the irradiation dose may be between 0, 0.5, 1, 1.5, 2, 2.5, 3, 3.5, 4, 4.5, 5, 5.5, 6, 6.5, 7, 7.5, 8, 8.5, 9, 9.5, or 10 and/or any value therebetween.

Lactic acid bacteria strains of the present invention may secrete/produce an antimicrobial compound such as a bacteriocin. In an embodiment of the present invention, Lactococcus lactis subsp. lactis MM19 accession number NML-080508-01 strain may produce/secrete the bacteriocin nisin. Lactococcus lactis subsp. lactis MM19 accession number NML-080508-01 strain may produce/secrete the bacteriocin nisin encoded by the DNA sequence of SEQ ID NO.:6. In an embodiment of the present invention, Pediococcus acidilactici MM33 accession number NML-080508-02 may produce/secrete the bacteriocin pediocin. Pediococcus acidilactici MM33 accession number NML-080508-02 may produce/secrete the bacteriocin pediocin encoded by the protein sequence of SEQ ID NO.:1. The present invention also encompasses an isolated non-bacteriocin lactic acid bacteria having the identifying characteristics of strain MM33A.

The lactic acid bacteria strains of the invention may inhibit the growth of a broad range of gram-positive bacteria. The lactic acid bacteria strains of the invention may inhibit the growth of antibiotic-resistant gram-positive bacteria. The lactic acid bacteria strains of the invention may inhibit the growth of a vancomycin resistant enterococcus bacteria (for example, Enterococcus faecium). The lactic acid bacteria strains of the present invention may have been obtained from human intestines (from human fecal matter; from human stools; from the human gut) and/or have the ability to grow in and/or colonize human intestines.

The present invention also relates to a (substantially) cell-free culture supernatant (CFS) of a (pure) culture of an isolated lactic acid bacteria that may have the identifying characteristics of a strain selected from Lactococcus lactis subsp. lactis MM19 and/or Pediococcus acidilactici MM33. The (substantially) cell-free culture supernatant of the present invention may comprise an antimicrobial compound (bacteriocin) produced/secreted by the lactic acid bacterial strains of the present invention grown in culture.

A (substantially) “cell-free culture supernatant” may be obtained by separating the lactic acid bacterial cells from a (pure) culture of the invention by any separation means known to a person skilled in the art. In an exemplary embodiment of the present invention, the lactic acid bacterial cells may be separated from the (pure) culture by centrifuging the (pure) culture (for e.g. at 6000 g for 30 minutes at 4° C.) and recuperating the supernatant. Alternative means, ways and devices designed to separate and/or recuperate soluble and insoluble fractions, whether they are manual or automated, are also within the scope of the invention. The obtained CFS may be used without any further isolation and/or purification steps (crude CFS). A “substantially” cell-free culture supernatant encompasses a culture supernatant that may be between 85-100% cell-free, for example, more than 85% cell-free, more than 90% cell-free, more than 95% cell-free, more than 98% cell-free and/or more than 99% cell-free.

Once obtained, the (substantially) cell-free culture supernatant may be filtered (for e.g. using a 0.2 μm filter). Filtration may ensure complete removal of any remaining bacterial cells and/or bacterial cell debris. In an exemplary embodiment of the present invention, the cell-free supernatant may also be concentrated. The cell-free culture supernatant may be neutralized. The cell-free supernatant may be concentrated and neutralized. Neutralization refers to the addition of any suitable substance to the cell-free supernatant so as to achieve a final pH ranging from between 6.5 to 7.5, including any value therebetween. Any concentrating means known to a person skilled in the art to concentrate the CFS and its low-molecular weight protein compounds are within the scope of the present invention. For example, Sep-Pak separation and/or rotavapor may be used to concentrate the cell-free supernatant.

An isolated bacteriocin produced by an isolated lactic acid bacteria that may have the identifying characteristics of a strain selected from Lactococcus lactis subsp. lactis MM19 and/or Pediococcus acidilactici MM33 is also encompassed in the present invention. Any of the several means known in the art of protein purification to isolate a proteinaceous compound such as a bacteriocin are encompassed in the present invention. Various methods of purification are described in, for example, “Protein Purification Protocols” by Paul Cutler. In an exemplary embodiment of the present invention, the bacteriocin may be purified from a culture and/or a cell-free culture supernatant obtained from Lactococcus lactis subsp. lactis MM19 and/or Pediococcus acidilactici MM33.

In an additional aspect thereof, the present invention relates to a composition that may comprise (consist of; consist essentially of) the isolated lactic acid bacteria of the present invention (Lactococcus lactis subsp. lactis MM19 accession number NML-080508-01 and/or Pediococcus acidilactici MM33 accession number NML-080508-02), the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and a carrier. Combination of the above may include, for example and without limitation, a composition that may comprise (consist of; consist essentially of) the isolated lactic acid bacteria of the present invention (Lactococcus lactis subsp. lactis MM19 accession number NML-080508-01 and/or Pediococcus acidilactici MM33 accession number NML-080508-02) in combination with the isolated bacteriocin of isolated lactic acid bacteria of the invention and a carrier.

Compositions of the present invention may be (used) for inhibiting microbial growth, for modulating the gut flora in a mammal, for preventing and/or treating a microbial infection in a mammal and/or for preventing and/or reducing the level of microbial colonization in a food product.

Encompassed in the present invention are compositions that may comprise (consist of; consist essentially of) the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and a carrier for use in the prevention and/or treatment of a microbial infection.

A “carrier” in a composition of the invention may be used, for example and without limitation, for solubilization, preservation, stabilization, emulsification, filling, coloring, odoring and/or antioxidative purposes. Carriers of the present invention may be aqueous and/or non-aqueous solutions. When compositions of the invention are administered to a mammal, the carriers in such compositions may be any type of carrier that have little or no negative and/or toxic side effects. In general, any carrier used in a composition of the present invention should not impact negatively on the function and/or use of the isolated lactic acid bacteria of the invention, the (pure) culture of isolated lactic acid bacteria of the invention, the cell-free culture supernatant (pure) culture of isolated lactic acid bacteria of the invention and/or the isolated bacteriocin present in such composition. A carrier in a composition of the invention may also encompass a nutritionally acceptable carrier such as any liquid and/or solid form of nourishment that a mammal may assimilate.

In a further aspect thereof, the present invention relates to a food product that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof.

According to the present invention, a “food product” refers to any substance that may be ingested by a mammal. Such food product may be, for example and without limitation, meat, dairy, fruit, vegetable, grain, cereal, alcohol, water and/or beverage products. The food product of the present invention may be fermented and/or non-fermented food products. In an exemplary embodiment of the present invention, a food product may be a fermented food product, for example a fermented dairy food product (for e.g. milk and/or cheese), a fermented soy food product, a fermented vegetable food product and/or a fermented meat food product (for e.g. salami, chorizo, dry and semi-dry sausages, pancetta, prosciutto, bacon, etc). The isolated lactic acid bacteria of the present invention may be constitutively present in the food product. For example, a lactic acid bacteria of the present invention may be used to ferment a dairy, a soy, a vegetable and/or a meat food product. The isolated lactic acid bacteria of the invention, the (pure) culture of isolated lactic acid bacteria of the invention, the cell-free culture supernatant (pure) culture of isolated lactic acid bacteria of the invention and/or the isolated bacteriocin of the invention may also be added to food products (food additive). For example, (concentrated) (neutralized) cell-free culture supernatant may be added to a food product. A food product that may comprise (consist of; consist essentially of) the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof for use in the prevention and/or treatment of a microbial infection is also encompassed herein.

In yet a further aspect thereof, the present invention relates to a method for inhibiting (reducing, decreasing, lowering, impairing) microbial growth. The method may comprise (consist of; consist essentially of) the step of contacting a microbe with the isolated lactic acid bacteria of the present invention, contacting a microbe with the (pure) culture of isolated lactic acid bacteria of the invention, contacting a microbe with the cell-free culture supernatant obtained from (pure) culture of isolated lactic acid bacteria of the invention, contacting a microbe with a composition of the present invention, contacting a microbe with the isolated bacteriocin of isolated lactic acid bacteria of the invention, contacting a microbe with the food product that may comprise the isolated lactic acid bacteria strain of the present invention and/or combination thereof. The contact may occur in a food product and/or in a mammal (in need thereof) and/or samples derived therefrom.

It is to be understood herein that by “inhibiting” microbial growth it is meant a process by which the microbial growth may be reduced, decreased, lowered and/or impaired. Inhibition may be partial and/or complete. Inhibition may occur at any time following contact.

“Contact” of a microbe in a food product may involve combining the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention, the composition of the invention and/or combination thereof with the food product and/or a sample derived therefrom wherein the food product and/or food product sample may comprise and/or may be suspected of comprising microbes.

Contacting a microbe in a sample derived from a mammal may include combining the isolated lactic acid bacteria of the present invention, the (pure) culture of isolated lactic acid bacteria of the invention, the cell-free culture supernatant obtained from (pure) culture of isolated lactic acid bacteria of the invention, the composition of the invention, the isolated bacteriocin of isolated lactic acid bacteria of the invention, the food product of the invention and/or combination thereof in vitro or ex vivo in a biological sample. As used herein, a biological sample refers to a sample obtained from biological fluids or tissues of a mammal; it is also meant to encompass derivatives and fractions of such samples (e.g., cell lysates). The biological sample may be suspected of comprising and/or may comprise microbes.

Contacting a microbe in a mammal may include administering an effective amount of the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention, the composition of the invention, the food product of the invention and/or combination thereof in vivo to a mammal in need thereof. As such, the present invention also relates to a method for inhibiting (reducing, decreasing, lowering, impairing) microbial growth that may comprise (consist of; consist essentially of) the step of administering an effective amount of a composition that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof. The method may also comprise the step of administering the food product of the invention to a mammal in need thereof.

The use of a composition that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and/or the use of a food product of the invention for the manufacture of a medicament for inhibiting (reducing, decreasing, lowering, impairing) microbial growth is encompassed within the present invention.

A composition that may comprise the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and/or the use of a food product of the invention for use in inhibiting (reducing, decreasing, lowering, impairing) microbial growth is also encompassed within the present invention.

It is to be understood that the term “microbe” as used herein, for example in microbial growth, microbial infection and/or microbial colonization, is meant to include any organisms comprised in the phylogenetic domains bacteria and archea. The term microbe may encompass, without limitation, pathogenic microbes (microbes that may cause a deleterious effect in a mammal such as, for example, eliciting a disease response), food-borne microbes (microbes that may grow in food products and/or may colonize and/or infect a mammal following food ingestion), antibiotic-resistant microbes (microbes that may have partially or completely reduced susceptibility to one or more antibiotics) and/or spoilage microbes (microbes that may cause food to deteriorate).

Microbes responsible for microbial growth, microbial infection and/or microbial colonization may encompass any gram-positive bacteria and/or gram-negative bacteria. Exemplary genus of gram positive bacteria encompassed within the invention may be the Enterococcus genus, Kocuria genus, Lactobacillus genus, Listeria genus, Pediococcus genus and Staphylococcus genus.

Exemplary gram-positive bacteria within the scope of the present invention may be Enterococcus faecalis, Enterococcus faecium, Kocuria varians, Lactobacillus rhamnosus GG, Lactobacillus acidophilus, Lactobacillus curvatus, Lactobacillus bulgaricus, Lactobacillus rhamnosus, Lactobacillus sakei, Listeria monocytogenes ½ a, Listeria monocytogenes ½ b, L. monocytogenes 4b, Pediococcus acidilactici, Pediococcus acidilactici MM33 and/or Staphylococcus aureus. The Lactobacillus rhamnosus strain may be ATCC 9595 and/or FRDC RW-9595M. The Listeria monocytogenes ½a strain may be HPB 1043, HPB 2569 and/or HPB 2812. The Listeria monocytogenes ½b strain may be HPB 2371, HPB 2558 and/or HPB 2739. The Listeria monocytogenes 4b strain may be HPB 1174 and/or HPB 2142.

The Lactococcus lactis subsp. lactis MM19 strain of the present invention may be able to inhibit the growth of and/or prevent or treat an infection caused by and/or prevent or reduce colonization by the following gram-positive bacteria: Enterococcus faecium, Kocuria varians, Lactobacillus rhamnosus GG, Lactobacillus acidophilus, Lactobacillus curvatus, Lactobacillus bulgaricus, Lactobacillus rhamnosus, Lactobacillus sakei, Pediococcus acidilactici, Pediococcus acidilactici MM33 and/or Staphylococcus aureus. The Lactobacillus rhamnosus strain may be ATCC 9595 and/or FRDC RW-9595M.

The Pediococcus acidilactici MM33 strain of the present invention may be able to inhibit the growth of and/or prevent or treat an infection caused by and/or prevent or reduce colonization by the following gram-positive bacteria: Enterococcus faecalis, Enterococcus faecium, Lactobacillus rhamnosus GG, Lactobacillus curvatus, Lactobacillus rhamnosus, Lactobacillus sakei, Listeria monocytogenes ½a, Listeria monocytogenes ½b, L. monocytogenes 4b, Pediococcus acidilactici, and/or Staphylococcus aureus. The Lactobacillus rhamnosus strain may be ATCC 9595. The Listeria monocytogenes ½a strain may be HPB 1043, HPB 2569 and/or HPB 2812. The Listeria monocytogenes ½b strain may be HPB 2371, HPB 2558 and/or HPB 2739. The Listeria monocytogenes 4b strain may be HPB 1174 and/or HPB 2142.

Exemplary antibiotic resistant microbes of the present invention may be antibiotic-resistant gram-positive bacteria. Antibiotic classes to which Gram-positive bacteria may develop resistance include, for example, the penicillins (e.g., penicillin G, ampicillin, methicillin, oxacillin, and amoxicillin), the cephalosporins (e.g., cefazolin, cefuroxime, cefotaxime, ceftriaxone and ceftazidime), the carbapenems (e.g., imipenem, ertapenem and meropenem), the tetracyclines and glycylcylines (e.g., doxycycline, minocycline, tetracycline, and tigecycline), the aminoglycosides (e.g., amikacin, gentamycin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid), and the lipopeptides (e.g., daptomycin). An exemplary antibiotic-resistant microbe (gram-positive bacteria) of the present invention may be, without limitation, vancomycin resistant enterococcus (VRE).

The present invention also encompasses the use of isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention, the composition of the invention, the food product of the present invention and/or combination thereof for inhibiting (reducing, decreasing, lowering, impairing) microbial growth.

In an additional aspect thereof, the present invention relates to a method for modulating the gut flora in a mammal in need thereof. The method may comprise (consist of; consist essentially of) the step of administering an effective amount of a composition that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof. The method may comprise (consist of; consist essentially of) the step of administering a food product that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof.

“Gut flora” is meant to refer to the microbial flora (microbiota) which normally inhabits the human gut. “Modulating” the gut flora encompasses either an increase and/or a decrease in the development (such as the growth) of the gut flora, whichever is advantageous to the host. For example, modulation may involve decreasing, suppressing, attenuating, diminishing and/or arresting the development of deleterious gut flora. In an exemplary embodiment of the present invention, the deleterious flora may comprise bacteria of the Staphylococcus and/or Enterobacteria genus. Modulation may also comprise promoting, increasing, intensifying and/or augmenting the development of beneficial flora. In an exemplary embodiment of the present invention, the beneficial flora may comprise Lactobacilli and/or lactic acid bacteria.

As used herein, an “effective amount” is the necessary quantity to obtain positive results without causing excessively negative effects in the host to which the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria, the composition of the invention, the food product of the invention and/or combination thereof may be administered. An exemplary effective amount encompassed in the present invention relate to a quantity which may be sufficient to inhibit microbial growth. An effective amount may also encompass an amount sufficient to prevent the establishment of an infection and/or substantially improve some symptoms associated with an infection. An effective amount may also encompass an amount sufficient to modulate the gut flora. An effective amount may also encompass a quantity which may be sufficient to prevent and/or reduce in any manner the growth and/or colonization of microbes in a food product.

An effective amount may be administered in one or more administrations, according to a regimen. The privileged method of administration and the quantity that may be administered is function of many factors. Among the factors that may influence this choice are, for example, the condition, the age and the weight of the host to which a composition is to be administered. An exemplary form of administration of the present invention may be oral administration. Oral administration may comprise any food forms (food products) and/or any food supplements including, but not limited to, capsules, tablets, liquid bacterial suspensions, dried oral supplements, wet oral supplements, dry tube feeding and/or wet tube feeding. Isolated lactic acid bacteria of the present invention may be administered in a lyophilised form.

The present invention also encompasses the use of a composition that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof for modulating the gut flora (in a mammal; for a mammal). The use of a food product that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof for modulating the gut flora (in a mammal; for a mammal) is also encompassed by the present invention.

In yet an additional aspect thereof, the present invention relates to a method for preventing and/or treating a microbial infection in a mammal in need thereof. The method may comprise (consist of; consist essentially of) administering an effective amount of a composition that may comprise (consist of; consist essentially of) the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof and a carrier to a mammal in need thereof. The method may also comprise (consist of; consist essentially of) administering a food product that may comprise (consist of; consist essentially of) the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof to a mammal in need thereof.

A “microbial infection” refers to the multiplication and/or colonization of a microbe in an individual's body tissues. In an exemplary embodiment, an infection may be caused by a pathogenic microbe but an “infection” is meant to encompass the multiplification and/or colonization of non-pathogenic microbes as well. An exemplary infection encompassed herein may be a bacteremia (infection caused by bacteria; a bacterial infection). An infection may be asymptomatic (clinically unapparent) or symptomatic. An exemplary infection may be a gastro-intestinal infection. Exemplary infections of the invention may be, for example and without limitation, infections caused by Listeria bacterial species (listeriosis), Enterococcus bacterial species (an enterococcal infection), Kocuria bacterial species and/or Staphylococcus bacterial species.

By “preventing” an infection, it is meant a process by which an infection may be prevented from establishing itself (occurring) within a mammal. For example, by arresting/inhibiting the colonization and/or development of microbes, an infection may be prevented. By “treating” an infection it is meant a process by which the development and/or colonization of microbes causing the infection is reduced either partially or totally. Treating an infection also encompasses a process by which the symptoms of an infection may not worsen, may remain stable, may be reduced and/or may be completely eliminated.

The present invention also encompasses the use of a composition that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof for preventing and/or treating a microbial infection (in a mammal). The use of a food product that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof for preventing and/or treating a microbial infection (in a mammal) is also encompassed by the present invention.

The present invention also encompasses the use of a composition that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof in the manufacture of a medicament for preventing and/or treating a microbial infection (in a mammal). The use of a food product that may comprise (consist of; consist essentially of) the isolated lactic acid bacteria of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof in the manufacture of a medicament for preventing and/or treating a microbial infection (in a mammal) is also encompassed by the present invention.

In a further aspect thereof, the present invention relates to a method for preventing and/or reducing (decreasing, lowering, impairing) the level of microbial colonization in a food product. The method may comprise (consist of; consist essentially of) contacting the food product with an effective amount of a composition that may comprise (consist of; consist essentially of) the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof.

“Microbial colonization” (contamination) in a food product refers to the growth of microbes that may be deleterious to the food product or to a mammal that may ingest the food product. A deleterious microbe in a food product may be a microbe that prematurely leads to spoilage of the product. A deleterious microbe for a mammal may be a microbe that upon ingestion of the food product will lead to an deleterious effect in a mammal (such as a food pathogen).

By “preventing colonization” it is meant preventing the growth of microbes (for example, pathogenic microbes, food-borne microbes, antibiotic-resistant microbes and/or spoilage microbes) within a food product. By “reducing colonization” is meant to reduce, decrease, lower and/or impair the growth of microbes within a food product. By preventing and/or reducing colonization in a food product, the shelf-life of a food product may be increased.

The use of a composition that may comprise (consist of; consist essentially of) the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention and/or combination thereof for preventing and/or reducing the level of microbial colonization in a food product is also encompassed in the present invention.

In an exemplary embodiment of the present invention, a mammal may be a human being. A mammal in need of preventing and/or treating a microbial infection may be a mammal having or suspected of having a microbial infection. Such mammal may or may not present symptoms of a microbial infections. A mammal in need of modulating its gut flora may be a mammal for which an increase and/or a decrease in the development (such as the growth) of its gut flora, whichever is advantageous, would be beneficial.

In an eight aspect thereof, the present invention relates to a kit. The kit may comprise (consist of; consist essentially of) at least one container that may contain the isolated lactic acid bacteria strain of the present invention, the (pure) culture of isolated lactic acid bacteria of the invention, the cell-free culture supernatant obtained from (pure) culture of isolated lactic acid bacteria of the invention, the isolated bacteriocin of isolated lactic acid bacteria of the invention, the composition of the invention, the food product of the invention and/or combination thereof. A kit of the present invention may also comprise instructions for its use in the form of a pamphlet or of any other support, indicating, for example, the instructions for the administration of the product contained therein and/or the instructions to mix given components.

Further scope and applicability of the present invention will become apparent from the detailed description given hereinafter. It should be understood, however, that this detailed description, while indicating exemplary embodiments of the invention, is given by way of example only, since various changes and modifications will become apparent to those skilled in the art.

### EXAMPLES

### Example 1

### Isolation and Identification of Bacteriocin Producing Strains

### Isolation of Bacteriocin-Producing Bacteria

Bacteriocin-producing bacteria were isolated by the direct plating method. Briefly, 25 g of healthy human stool sample was mixed with 225 ml of 0.85% (w/v) of peptone water and serial tenfold dilution were prepared in the same diluent. Plates of Lactobacilli MRS agar were spread with 0.1 ml of each dilution and were incubated at 36±1° C. for 24 h under anaerobic conditions. A total of 111 nonmotile, gram-positive, catalase- and oxidase-negative cocci isolates, harvested from two stool samples obtained from a single human male (30 years old) were examined. From these 111 colonies, seven of them scored positive as they produced clear zones of inhibition against at least one indicator strain on agar media. Two were selected and designated MM19 and MM33.

### Micro-Organisms and Growth Conditions

The indicator bacterial strains used are listed in TABLE 1. All bacteria were maintained at −80° C. in appropriate media containing 10% glycerol (w/v). All isolated and indicator strains of LAB were propagated in their respective culture broth as indicated in TABLE 1 at 36±1° C. Before being used in experiments, strains were propagated twice in broth overnight. Soft agar media was prepared by the addition of 0.75% (w/v) instead of 1.5% agar to liquid culture to examine the antimicrobial capacity of given bacteria by the well-diffusion assay.

One liter of Lactobacilli MRS broth was inoculated with 10 ml of L. lactis subsp. lactis MM19 or P. acidilactici MM33 and incubated for 24 h at 36±1° C. Cell-free supernatant or cell-free culture supernatant (CFS) was obtained by centrifuging the culture at 6000 g for 30 minutes at 4° C. followed by neutralization to pH 6.5 by the addition of 5 mol l⁻¹ of NaOH. The resulting CFS was then filtered through a 0.2 μm filter (Sarstedt, Montreal, QC, Canada) to remove any remaining bacterial cells.

### Activity Determination Assay

Screening of bacteriocin production was performed by the agar well diffusion assay as described by Schillinger and Lucke (1989). Liquid lactobacilli MRS agar was inoculated with 10⁷ CFU ml⁻¹ of one indicator strain, poured into 100×15-mm standard Petri dishes and allowed to solidify for 30 minutes at room temperature. Wells of 6 mm in diameter were cut and 80 μl of CFS of the bacteriocin-producing strains were placed into each well. All plates were then incubated at 36±1° C. for 24 h and examined for formation of inhibition zones. Inhibition was scored positive if the width of the clear zone around the well was ≧0.5 mm.

The inhibitory spectrum of each bacteriocin-producing strain is presented in TABLE 1. L. lactis subsp. lactis MM19 exhibited inhibitory activity against a broad range of bacteria in the genera Enterococcus, Kocuria, Lactobacillus, Pediococcus and Staphylococcus. P. acidilactici MM33 showed an inhibitory spectrum against Enterococcus, Lactobacillus, Listeria and Pediococcus. Interestingly, bacteriocin production was detected on solid media using crude CFS. This contrast with other reports wherein a concentration step was needed to demonstrate antimicrobial activities of bacteriocins (Carolissen-Mackay et al. 1997; Toure et al. 2003).

TABLE 1
INHIBITORY ACTIVITY OF NEUTRALIZED, CELL-FREE
SUPERNATANTS FROM CULTURES OF LACTOCOCCUS LACTIS
SUBSP. LACTIS MM19 AND PEDIOCOCCUS ACIDILACTICI
MM33 AGAINST INDICATOR MICROORGANISMS

| | | | Inhibition† | |
|---|---|---|---|---|
| Indicator microorganisms | Source* | Medium | MM19 | MM33 |
| Enterococcus faecalis | LSPQ 2724 | BHI | — | + |
| Enterococcus faecium | LSPQ 3550 | BHI | + | + |
| Escherichia coli | ATCC 25922 | BHI | — | — |
| E. coli 0157: H7 | EDL 933 | BHI | — | — |
| Kocuria varians | Our | MRS | + + | — |
| | collection | | | |
| Lactobacillus rhamnosus | ATCC 53103 | MRS | + + + | + + + |
| GG | | | | |
| Lactobacillus acidophilus | ATCC 4356 | MRS | + + | — |
| Lactobacillus curvatus | FRDC V32 | MRS | + + | + + |
| Lactobacillus bulgaricus | Private | MRS | + + + | — |
| | collection | | | |
| Lactobacillus rhamnosus | ATCC 9595 | MRS | + + + | + |
| Lactobacillus rhamnosus | FRDC RW- | MRS | + + + | — |
| | 9595M | | | |
| Lactobacillus sakei | ATCC 15521 | MRS | + + + | + + + |
| Lactococcus lactis subsp. | ATCC 11454 | MRS | — | — |
| lactis | | | | |
| Lactococcus lactis subsp. | Private | MRS | — | — |
| lactis MM19 | collection | | | |
| Listeria innocua | LSPQ 3285 | BHI | — | — |
| Listeria monocytogenes | HPB 1043 | BHI | — | + + + |
| 1/2 a | | | | |
| L. monocytogenes 4b | HPB 1174 | BHI | — | + + + |
| L. monocytogenes 4b | HPB 2142 | BHI | — | + + |
| L. monocytogenes 1/2 b | HPB 2371 | BHI | — | + + + |
| L. monocytogenes 1/2 b | HPB 2558 | BHI | — | + + + |
| L. monocytogenes 1/2 a | HPB 2569 | BHI | — | + + + |
| L. monocytogenes 1/2 b | HPB 2739 | BHI | — | + + + |
| L. monocytogenes 1/2 a | HPB 2812 | BHI | — | + + + |
| Pediococcus acidilactici | Our | MRS | + + | + |
| | collection | | | |
| P. acidilactici MM33 | Our | MRS | + + + | — |
| | collection | | | |
| Pseudomonas aeruginosa | ATCC 15442 | BHI | — | — |
| Pseudomonas fluorescens | FRDC V491 | BHI | — | — |
| Pseudomonas fragi | FRDC V378 | BHI | — | — |
| Pseudomonas putida | FRDC V376 | BHI | — | — |
| Salmonella choleraesuis | ATCC 19430 | BHI | — | — |
| subsp. choleraesuis | | | | |
| serotype Typhi | | | | |
| Salmonella Typhimurium | SL1344 | BHI | — | — |
| Serratia liquefaciens | Our | BHI | — | — |
| | collection | | | |
| Staphylococcus aureus | ATCC 29213 | BHI | + + | — |

*ATCC—American Type Culture Collection, Rockville, MD, USA; LSPQ—Laboratoire de Santé Publique, Ste-Anne-de-Bellevue, QC, Canada; FRDC—Food Research and Development Center, St-Hyacinthe, QC, Canada; HPB—Health Product Branch, Santé, Canada.
†+ + +, diameter of the inhibition zone ≧20 mm; + +, 10-19 mm; +, 7-9 mm; —, no inhibition.

### Identification of MM19 and MM33 Strains

Gram staining, motility, catalase and oxidase tests were conducted as a preliminary step for characterization of selected bacteria. In order to precisely identify given species, DNA extraction and PCR amplification of DNA coding for 16S ribosomal RNA (16S rDNA) procedures were performed. The sequences of the 16S rDNA of isolates MM19 and MM33 were compared with DNA sequences from the National Center for Biotechnology Information (NCBI) database using the standard nucleotide-nucleotide homology search Basic Local Alignment Search Tool (Altschul et al. 1990). 16S rDNA PCR products from MM19 and MM33 isolates showed 98% and 97% homology with that of L. lactis subsp. lactis and P. acidilactici respectively, thereby identifying the MM19 and MM33 isolates.

### Antibiotic Resistance Profile of MM19 and MM33 Strains

The protocol was adapted from Delgado et al. (2005). Minimum Inhibitory Concentration (MIC) Plate (# GPN3F) and Anaerobe MIC Plate (# AN02B) were used to determine the antibiotic sensitivity and resistance profile of MM19 and MM33 according to the instructions of the manufacturer (Trek Diagnostic Systems; Nova Century, Burlington, ON, Canada).

Lactococcus lactis subsp. lactis MM19, Pediococcus acidilactici MM33 and Lactobacillus rhamnosus GG (ATCC 53103) from frozen glycerol stock were first inoculated in Lactobacilli MRS and incubated at 37° C. for 18 h. 100 μl of this culture were further incubated under the same conditions. This second culture was then inoculated on a MRS agar plate and incubated at 37° C. for 48 h under anaerobic conditions using GasPak Plus system (BD BBL, Sparks, Md., USA). Results are shown in TABLE 2.

TABLE 2
ANTIBIOTIC MICs OF L. LACTIS
MM19 AND P. ACIDILACTICI MM33

| | | MIC^{a} (μg/ml) | | |
|---|---|---|---|---|
| | | L. rhamnosus | | |
| Plate | Antibiotics | GG | MM19 | MM33 |
| ANO2^{b} | A/S | 1/0.5 | <0.5/0.25 | 4/2 |
| | AUG | 1/0.5 | <0.5/0.25 | 4/2 |
| | AMP | 1 | <0.5 | 4 |
| | TANS | >64 | >64 | >64 |
| | FOX | >32 | >32 | >32 |
| | CHL | 8 | 4 | 16 |
| | CLI | 0.5 | 0.5 | <0.25 |
| | IMI | 2 | 0.25 | 0.5 |
| | MERO | 8 | <0.5 | 2 |
| | MRD | >16 | >16 | >16 |
| | MEZ | <4 | <4 | 8 |
| | PEN | 0.5 | 0.5 | 1 |
| | PIP | <4 | <4 | 8 |
| | P/T | 1/4 | 2/4 | 8/4 |
| | TET | 1 | 0.375 | >8 |
| GPN3F^{c} | ERY | <0.25 | <0.25 | <0.25 |
| | CLI | <0.12 | 0.25 | <0.12 |
| | SYN | 1-2 | 4 | 1 |
| | DAP | 2 | 0.5-1 | 1 |
| | VAN | >128 | <1 | >128 |
| | TET | <2 | <2 | >16 |
| | AMP | 1 | 0.12-0.25 | 2 |
| | GEN | <2 | 2-4 | <2 |
| | LEVO | 2-4 | 0.5 | >8 |
| | LZD | 1-2 | 2 | 4-8 |
| | AXO | 32-64 | <8 | 16 |
| | STR | <1000 | <1000 | <1000 |
| | PEN | 0.5 | 0.25-0.5 | 0.5 |
| | RIF | <0.5 | >4 | 0.5-1 |
| | GAT | <1 | <1 | <1 |
| | CIP | 1 | 2 | >2 |
| | SXT | >4/76 | >4/76 | >4/76 |
| | OXA+ | 0.25-0.5 | 1 | 2 |

^{a}Median of two repetitions
^{b}A/S: Ampicillin/Sulbactam; AUG: Amoxicillin/Clavulanic acid; AMP: Ampicillin; TANS: Cefotetan; FOX: Cefoxitin; CHL: Chloramphenicol; CLI: Clindamycin; IMI: Imipenem; MERO: Meropenem; MRD: Metronidazole; MEZ: Mezlocillin; PEN: Penicillin; PIP: Piperacillin; P/T: Piperacillin/Tazobactam; TET: Tetracycline
^{c}ERY: Erythromycin; CLI: Clindamycin; SYN: Quinupristin/Dalfopristin; DAP: Daptomycin; VAN: Vancomycin; TET: Tetracycline; AMP: Ampicillin; GEN: Gentamicin; LEVO: Levofloxacin; LZD: Linezolid; AXO: Ceftriaxone; STR: Streptomycin; PEN: Penicillin; RIF: Rifampin; GAT: Gatofloxacin; CIP: Ciprofloxacin; SXT: Trimethoprim/Sulfamethoxazole; OXA+: Oxacillin + 2% NaCl

### GI Tract Resistance of MM19 and MM33 Lab

Many potential probiotics do not resist, survive or temporarily colonize the intestine. Bile salt and acidic environment resistance is considered one of the most important attribute required by LAB to survive in the stomach, duodenum and the upper small intestine. LAB that would show such resistance have great potential. The resistance levels to bile salts and acidic environment of MM19 and MM33 strains were analyzed.

### Bile Salt Resistance of Lab

The bile salt resistance of LAB was ascertained in MRS agar containing a commercial preparation of bile salts. Bile salts mixture (Sigma B-3426) was added in concentration varying between 0 and 10% with increment of 1% or Bile Salts (LP0055, Oxoid, Nepean, ON, Canada) was tested in concentration from 0 to 24% with increments of 4%. Bile salts containing-MRS agar was autoclaved for 15 minutes at 121° C., cooled and finally plated. Overnight MRS broth culture (100 μl of bacteria in the stationary phase of growth) were inoculated on surface of bile salts-containing MRS agar and incubated at 37° C. for 72 h under anaerobic conditions. Presence of a bacterial lawn indicated good growth and thus good resistance of bacteria to bile salts while presence of small and isolated colonies indicated a poor resistance to bile salts. Absence of colony indicated that LAB did not tolerate the bile salt concentration assayed. Minimal inhibitory concentration represented the lowest concentration of the bile salts needed to totally inhibit the growth of colonies as judged by visual examination. Bile salt resistance of both LAB was compared to probiotic bacteria Lactobacillus rhamnosus GG ATCC 53103, an acid and bile resistant probiotic bacteria used as a positive control during gastrointestinal experiments. The results showed that bile salts mixture (Sigma) resistance threshold was 4% for all bacteria, while the pediococci grew in MRS containing 20% (w/v) of bile salts (Oxoid) as compared to 16% for L. rhamnosus GG YY and to 12% for L. lactis subsp. lactis MM19 (TABLE 3). All LAB survived to a bile salt stress but also grew on MRS agar containing 4% of a standardized bile salts mixture (Sigma) or higher concentrations of bile salts from Oxoid. The same experiments were performed using a bile salts mixture obtained from Oxgall and showed a MIC for L. lactis subsp. lactis MM19 of 9% and that of P. acidilactici MM33 more than 10%.

TABLE 3
MAXIMAL CONCENTRATION TOLERATED
(%) OF BILE SALT BY LAB

| | % Bile salts | % Bile salts mixture |
|---|---|---|
| Microorganisms | (Oxoid L55) | (Sigma B-3426) |
| Lc. lactis MM19 | 12 | 4 |
| P. acidilactici MM33 | 20 | 4 |
| L. rhamnosus GG | 16 | 4 |

### Acid Resistance of Lab

Simulated gastric fluid (SGF) was formulated according to United States Pharmacopea (USP). Briefly, SGF was composed by 3.2 g/l of pepsin (Sigma), 2.0 g/l NaCl and the pH was adjusted to 1.5, 2.0, 2.5 or 3.0 by addition of HCl (5 M). A volume of 1 ml of overnight MRS broth cultures of LAB were added in 19 ml of SGF for 30 minutes at 37° C. under mild agitation (200 rpm) in a G24 Environmental Incubator Shaker (New Brunswick Scientific Co. Inc., NJ, USA). After 30 minutes in gastric solution, 1 ml was collected, mixed in sterile phosphate buffer saline (PBS; pH 7.4) and immediately diluted sterile peptone water (0.1% p/v) and plated (pour-plate method) on Lactobacilli MRS agar. Plates were incubated under aerobic conditions at 37° C. for 48 h. The average number of CFU from triplicate analysis was determined by Darkfield Quebec Colony Counter. A similar process was carried out for bacteria without SGF treatment in order to determine the initial concentration of LAB. L. rhamnosus GG was used as a positive control.

Results presented in TABLE 4 show that P. acidilactici MM33 and L. rhamnosus GG survived under an acidic environment during 30 minutes. No significant difference (P>0.05) was observed between initial microbial population at 0 and 30 minutes under a pH≧2.5 for L. rhamnosus GG. However, a significant reduction of viability was observed at pH 2 and no viability was detected after 30 minutes at pH 1.5 for both bacteria. P. acidilactici MM33 survived well after an acidic treatment for 30 minutes at pH 2.5 but a one log decrease was observed as compared to initial enumeration (P≦0.05). Lactococcus lactis subsp. lactis MM19 showed mortality rates at pH≦2.5; however, complete survival was observed at pH 3.

TABLE 4
SURVIVAL OF LAB AFTER AN INCUBATION OF 30 MINUTES AT
37° C. IN SIMULATED GASTRIC FLUID (pH 1.5 TO 3.0)

| Microorganisms | Time (min) | pH | Log CFU survivor |
|---|---|---|---|
| Lc. lactis MM19 | 0 | — | 8.91 ± 0.21^{B}* |
| | 30 | 1.5 | <1 |
| | | 2.0 | 4.47 ± 0.46^{A} |
| | | 2.5 | 4.69 ± 0.71^{A} |
| | | 3.0 | 8.67 ± 0.26^{B} |
| P. acidilactici MM33 | 0 | — | 9.62 ± 0.10^{C} |
| | 30 | 1.5 | <1 |
| | | 2.0 | 4.7 ± 0.33^{A} |
| | | 2.5 | 8.62 ± 0.26^{B} |
| | | 3.0 | 9.54 ± 0.14^{C} |
| L. rhamnosus GG | 0 | — | 9.10 ± 0.13^{B} |
| | 30 | 1.5 | <1 |
| | | 2.0 | 5.33 ± 0.62^{A} |
| | | 2.5 | 9.08 ± 0.14^{B} |
| | | 3.0 | 9.01 ± 0.13^{B} |

*Different letters indicate a significant variation (P ≦ 0.05) as compared to the control (0 min) for each LAB assayed.

Results obtained demonstrated that both LAB completely resists to a simulated gastric fluid at pH≧3 and resist at a certain degree in variable pH. For example, P. acidilactici can tolerate a pH of 2.5.

### Example 2

### Characterization of MM19 and MM33 Antimicrobial Activity

Antimicrobial activities were tested using the agar well diffusion assay. To quantify the bacteriocin activity, CFS was serially diluted with sterile deionized water and 80 μl of each dilution were added into the wells. Lactobacillus sakei was used as the indicator strain. The antimicrobial activity was defined as the reciprocal of the highest dilution, which exerted total inhibition of the indicator lawn and was expressed in activity units (AU) per milliliter. The residual activity was calculated in comparison with neutralized CFS from strain MM19 or MM33, which corresponded to a 100% antimicrobial activity.

### Temperature Sensitivity

The thermostability of the antimicrobial activities was determined by heating neutralized CFS prepared from L. lactis subsp. lactis MM19 or P. acidilactici MM33 at 30-121° C. for 15, 30 or 60 minutes.

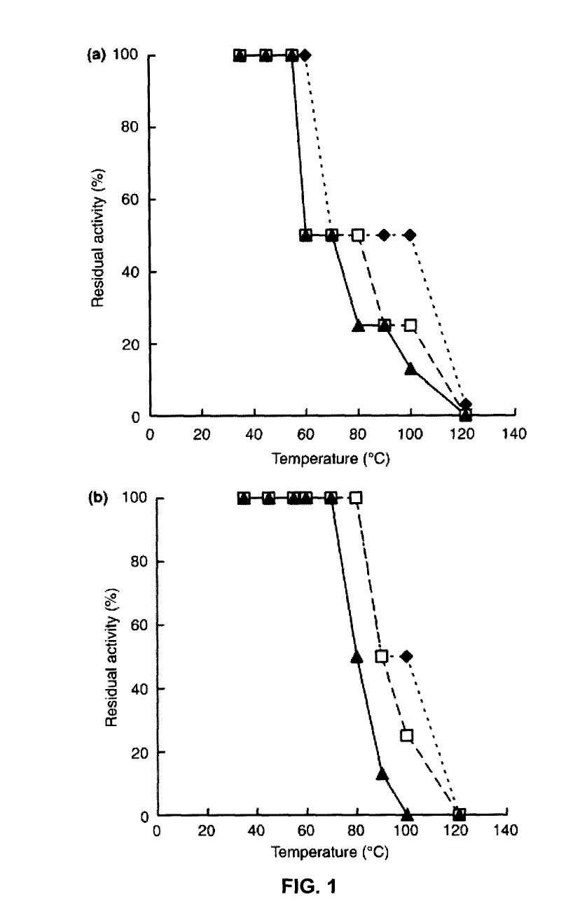

The effect of temperature on the antimicrobial activity is shown in FIG. 1. Lactococcus lactis subsp. lactis MM19 bacteriocin activity remained unchanged after incubation at 50° C. for 60 minutes. Some loss of activity was observed after incubation at 60° C. for 30 minutes or at 70° C. for 15 minutes. At 100° C., the residual antimicrobial activity decreased to 50%, 25% and 0% after 15, 30 and 60 minutes. Heating to 121° C. for 15 minutes inactivated the bacteriocin activity of L. lactis subsp. lactis MM19 (FIG. 1A). For P. acidilactici MM33, a decreased activity was observed after incubation at 80° C. for 60 minutes, at 90° C. for 30 minutes or at 100° C. for 15 minutes (FIG. 1B).

### pH Sensitivity

The effect of pH on the activity was determined by adjusting the pH of the CFS to values from pH2 to pH10 using 5 mol l⁻¹ of HCl or NaOH. The activity of each sample was compared with the activity of CFS at pH 6.5 or 5 for supernatant from L. lactis subsp. lactis MM19 or P. acidilactici MM33, respectively.

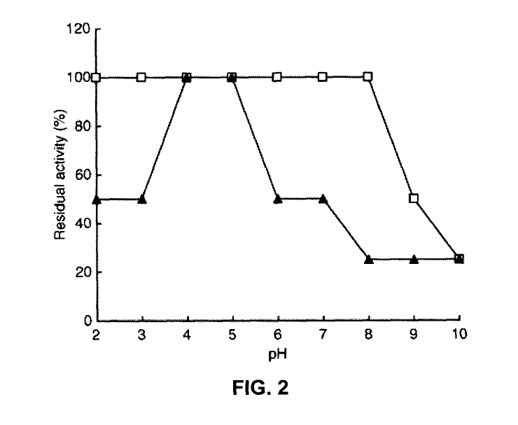

The effect of pH on the bacteriocin antimicrobial activity is shown in FIG. 2. The bacteriocin secreted by L. lactis subsp. lactis MM19 was stable after incubation for 2 h in solution with pH values ranging from 2 to 10. However, its levels of activity were somewhat reduced by incubation at pH 9 and 10. The bacteriocin produced by P. acidilactici MM33 remained fully active at pH 4 and 5, showed decreased activity at pH 3 and below and pH 6 and above.

### Proteases and Other Agents Sensitivity

The sensitivities of the antimicrobials to proteases or other agents was tested by incubating neutralized CFS in the presence of 1% α-chymotrypsin, pepsin, pronase E, trypsin, lipase, catalase, Tween-80, Triton X-100, urea, sodium dodecyl sulfate (SDS) or 0.25% proteinase K for 2 h at 36±1° C.

The effects of enzymes, detergents and other compounds on the CFS antimicrobial activity against L. sakei are presented in TABLE 5. Inhibitory activities were significantly decreased after protease treatments. P. acidilactici MM33 antimicrobial activity was also decreased by protease treatments. L. lactis subsp. lactis MM19 showed some residual antagonistic activity after trypsin and pepsin treatments. Lipase reduced the antimicrobial activity of L. lactis subsp. lactis MM19, but no inhibition of the bacteriocin-containing CFS secreted by P. acidilactici MM33 was observed. Catalase did not affect the activity of either type of CFS. Detergents doubled the inhibitory activity of the CFS obtained from P. acidilactici MM33. Urea reduced the activity of the CFS from L. lactis subsp. lactis MM19 but did not affect the activity of CFS from P. acidilactici MM33. Controls were done to verify the antimicrobial potential of each enzyme and other agents assayed during this work and none demonstrated antimicrobial activity. The antimicrobial activities of the CFS of L. lactis subsp. lactis MM19 and P. acidilactici MM33 were inhibited by proteases but not by catalase, showing the proteinaceous nature of the antimicrobials.

TABLE 5
EFFECTS OF VARIOUS ENZYMES, DETERGENTS AND UREA
ON THE ANTIMICROBIAL ACTIVITIES OF NEUTRALIZED
CFS FROM CULTURES OF L. LACTIS SUBSP. LACTIS
MM19 AND P. ACIDILACTICI MM33

Residual activity (%)*

| Additive | L. lactis MM19 | P. acidilactici MM33 |
|---|---|---|
| None | 100 | 100 |
| Pronase E (1%) | 0 | 0 |
| Proteinase K (0·25%) | 1.6 | 0 |
| Pepsin (1%) | 12.5 | 0.8 |
| Trypsin (1%) | 50 | 0.8 |
| α-chymotrypsin (1%) | 1.6 | 0 |
| Catalase (1%) | 100 | 100 |
| Lipase (1%) | 25 | 100 |
| Tween-80 (1%) | 100 | 200 |
| Triton X-100 (1%) | 100 | 200 |
| SDS (1%) | 100 | 200 |
| Urea (1%) | 25 | 100 |

*Antimicrobial activities of supernatants without additives are 100%. Results are means of three individual assays with a standard deviation less than 5% about the mean.
SDS, sodium dodecyl sulphate.

### Irradiation Sensitivity

γ-irradiation sensitivities of the CFS antimicrobial activities were determined with increasing irradiation doses ranging from 0 to 40 kGy, using a Gamacell UC15-A apparatus (MDS-Nordion; Laval) having a dose rate of 23 kGy. Residual antimicrobial activities were tested using the well diffusion assay against L. sakei.

Results of the effects of γ-irradiation on the CFS are presented in TABLE 6. Bacteriocin activity of L. lactis subsp. lactis MM19 was reduced when the CFS was irradiated at a dose of 4 kGy and was eliminated by a dose of 16 kGy. The antagonistic activity of the CFS of P. acidilactici MM33 was more resistant to γ-irradiation as it showed residual antimicrobial activity after a dose of 12 kGy and some residual activity was still observed when the CFS was treated with 40 kGy.

TABLE 6
EFFECTS OF γ-IRRADIATION ON THE ANTIMICROBIAL
ACTIVITIES OF NEUTRALIZED CFS OF L. LACTIS MM19
AND P. ACIDILACTICI MM33

Residual activity (%)*

| Doses (kGy) | L. lactis MM19 | P. acidilactici MM33 |
|---|---|---|
| 0 | 100 | 100 |
| 4 | 3.1 | 100 |
| 8 | 1.2 | 50 |
| 12 | 0.4 | 50 |
| 16 | 0 | 25 |
| 20 | 0 | 12.5 |
| 24 | 0 | 12.5 |
| 28 | 0 | 12.5 |
| 32 | 0 | 12.5 |
| 36 | 0 | 12.5 |
| 40 | 0 | 6.3 |

*Results are means of three individual assays with a standard deviation less than 5% about the mean.

### Example 3

### Kinetics of Bacteriocin Production and Size Determination

### Kinetics

The kinetics of bacteriocin production was determined after inoculation of 1 liter of lactobacilli MRS broth with a 1% (v/v) solution of L. lactis subsp. lactis MM19 or P. acidilactici MM33, followed by an incubation at 36±1° C. under agitation (100 rev min⁻¹) without pH control. The initial pH of the culture broth was 6.5. Samples of 10 ml were removed from the cultures every 2 h and were analyzed for microbial numbers, bacteriocin activity and pH. Bacterial growth was followed by enumeration of bacteria on lactobacilli MRS agar incubated for 48 h at 36±1° C.

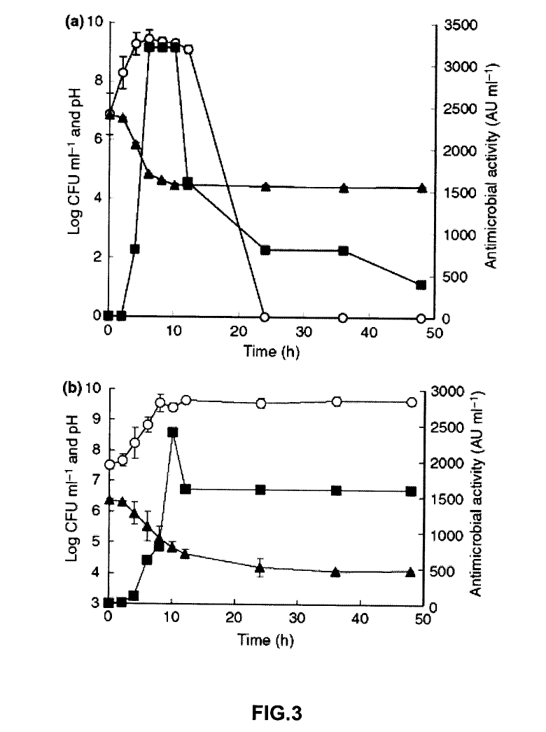

The production of bacteriocins in broth cultures by L. lactis subsp. lactis MM19 and P. acidilactici MM33 was maximal after 6 h and 10 h, respectively, where the maximum number of bacteria had been attained (FIG. 3). Minimum pH values of 4.4 and 4.1 were reached after 10 and 36 h with L. lactis subsp. lactis MM19 and P. acidilactici MM33, respectively. Bacteriocins production was maximal in the early stationary phase of growth and appeared to be growth-associated. Antimicrobial activity somewhat declined during the late stationary phase, and the L. lactis subsp. lactis MM19 population declined after 12 h of incubation.

### Size Determination

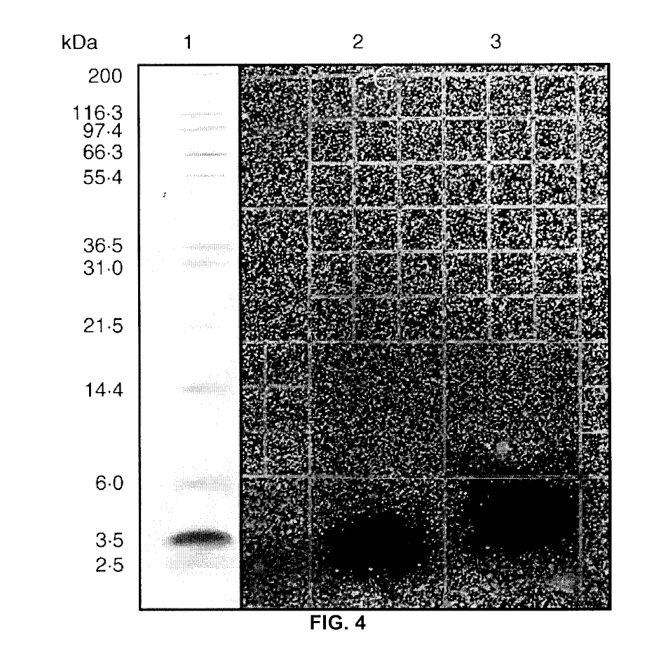

A volume of 20 μl of crude CFS from Lactococcus lactis subsp. lactis MM19 and P. acidilactici MM33 were analyzed with a NuPAGE 12% Bis-Tris gel kit (InVitrogen, Burlington, ON, Canada) ran at 200 V constant for 40 minutes. The molecular weight marker Mark 12 with a size range from 2.5 to 200-kDa kit (Invitrogen) was used. After electrophoresis, the first gel was stained with Coomassie Brilliant Blue R250 (Invitrogen). A duplicate gel was used for the plate overlay assay. The plate overlay assay was conducted to estimate the molecular weight of the bacteriocin (antimicrobial compounds). Briefly, a non-reduced SDS-PAGE gel pre-washed (sample not heated and without reducing agent) with sterile water was placed onto a plate of lactobacilli MRS agar and overlaid with lactobacilli MRS agar containing growing cells of Lact. sakei ATCC 15521 at numbers of about 10⁶ CFU ml⁻¹. The agar was allowed to solidify, then it was cooled at 4° C. for 60 minutes. After incubation for 18 h at 36±1° C., the formation of an inhibition zone indicated the position and size of active bacteriocin in the gel. With SDS-PAGE, smears of proteins were observed in samples from both micro-organisms when using Coomassie Brilliant Blue (results not shown). As shown in FIG. 4, inhibitory activities for both supernatants were detected as clear zones of inhibition between 3.5 and 6 kDa after gels were overlaid with Lact. sakei-seeded agar.

### Example 4

### Vancomycin-Resistant Enterococcus Inhibition In Vitro

### Bacterial Strains and Growth Conditions

A clinical isolate of vancomycin-resistant Enterococcus faecium (VRE) was provided by the Centre Hospitalier de l'Université de Montréal microbiology laboratory (CHUM; Montreal, QC, Canada). PCR studies using primers specific to the vancomycin-resistance genes have revealed that this strain is a VanA-type VRE. The E. faecium strain was maintained in brain-heart infusion (BHI; Difco) for E. faecium containing 10% glycerol (w/v).

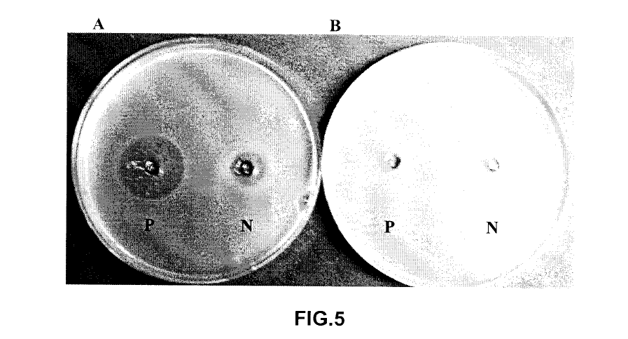

An agar well-diffusion assay was performed to verify the antimicrobial capacity of the neutralized cell-free supernatant (CFS) against a VRE clinical isolate. CFS was obtained as described before. A volume of 30 ml of cooled (45° C.) sterile BHI agar, containing 0.75% agar, was inoculated with 10⁷ colony-forming units (CFU)/ml of VRE, poured into a 15 mm standard Petri dishes and allowed to solidify for 30 minutes at room temperature. Wells of 6 mm in diameter were cut-out and 80 ml of antimicrobial agent were placed into each well. All plates were then placed at 4° C. for 30 minutes, incubated at 37° C. for 24 h and examined for inhibition zones. Inhibition was scored positive if the width of the clearing zone around the well was 0.5 mm. Antibiotics were also evaluated with this method to ascertain the resistance of the VRE strain. Vancomycin and clindamycin (Sigma-Aldrich, Oakville, ON, Canada) were used in concentrations ranging from 0 to 800 μg/ml. The results showed that the clinical isolate was resistant for all vancomycin concentrations evaluated between 0 to 800 μg/ml while the VRE strain was very sensitive to clindamycin at a concentration of 2 μg/ml (data not shown). When LAB supernatants were assayed, proteases from Streptomyces griseus type XIV (3 mg/ml; Sigma) were added in the soft agar in order to verify whether the inhibition is caused by bacteriocin. Results in FIG. 5 show a large inhibition zone of bacterial growth is related to a high potential of neutralized cell-free supernatant of LAB cultures to inhibit the growth of the antibiotic resistant VRE. Following proteases type XIV treatment in agar, no inhibition zone was observed.

### Example 5

### Purification and Sequencing of P. Acidilactici MM33 Bacteriocin

### Purification

The purification of the bacteriocin produced by P. acidilactici MM33 was performed using a modified version of the two-step procedure described by Uteng et al. (2002). CFS served as starting material for the purification procedures (fraction I). The bacterial culture supernatant was then loaded directly on a 20 ml HiPrep SP Fast Flow cation-exchange column (GE Healthcare) after equilibration with 50 mmol l⁻¹ acetate buffer, pH 5.0 (starting buffer) at a flow rate of 2.75 ml min⁻¹. After washing the column with 200 ml of starting buffer, the bacteriocin was eluted using a gradient (0-20% in 10 minutes; 20-30% in 120 minutes; 30-100% in 15 minutes) of elution buffer (50 mmol l⁻¹ acetate buffer containing 1 mol l⁻¹ NaCl, pH 5.0) at a flow rate of 2.75 ml min⁻¹. Fractions were then collected and tested using the well-diffusion assay for bacteriocin activity. The active fractions determined by 17 mm or less of inhibition zone following the antimicrobial activity test were pooled (pool 1) while the other active fractions (18 mm or more) were pooled separately (pool 2). Pooled fractions were then applied on 2 Sep-Pak Plus cartridges (Waters, Dorval, QC, Canada) in tandem to eliminate salts. Cartridges were first equilibrated with 20 ml of 100% acetonitrile (Laboratoire MAT, Beauport, QC, Canada) followed by 10 ml of water. Pooled sample was applied at a flow rate of 2 drops/s and successively washed with 10 ml of 0, 10, 20 and 30% acetonitrile and final elution was done with 30 ml of 80% acetonitrile. The 30 and 80% acetonitrile fractions were pooled and the solvent evaporated using a Rotavapor (Büchi, Switzerland) at 65° C. for 15 minutes (fraction III). The residual suspension was lyophilized overnight.

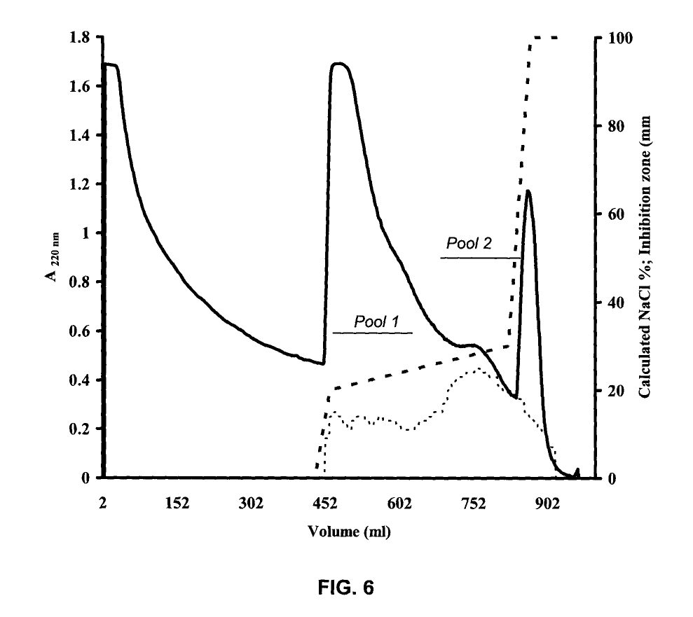

Cation exchange chromatography of the proteinaceous compound secreted by P. acidilactici MM33 is shown in FIG. 6. About fifty percent of total bacteriocin activity was recovered after cation-exchange chromatography in Fraction II with a specific activity 725-fold higher than that of the cell-free supernatant (CFS) (TABLE 7). After Sep-Pak separation and rotavapor concentration step, the purification yield was 40% with specific activity 5 725-fold higher than of the CFS.

TABLE 7
PURIFICATION OF BACTERIOCIN PRODUCED BY
PEDIOCOCCUS ACIDILACTICI MM33 (n = 2)

| | Vol- | Total | Total | Specific | Purifi- | |
|---|---|---|---|---|---|---|
| Purification | ume | activity | protein | activity | cation | Yield |
| steps | (ml) | (×10⁵ AU) | (mg) | (AU mg⁻¹) | fold | (%) |
| Culture | 500 | 12 | 18 748 | 64 | 1.0 | 100 |
| supernatant | | | | | | |
| HiPrep 16/10 | 167 | 6.08 | 13.1 | 46 412 | 725 | 50.7 |
| SP FF | | | | | | |
| Rotavapor | 7.8 | 4.8 | 1.31 | 366 412 | 5 725 | 40 |
| Freeze-dried | 1 | 6.08 | 0.26 | 2 338 462 | 36 539 | 50.7 |
| fraction | | | | | | |

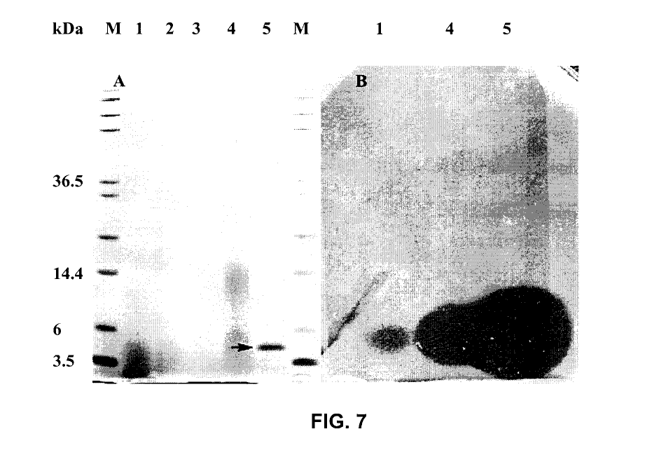

Separation by SDS-PAGE yielded a unique peptide band (black arrow in lane 5) between 3.5 and 6 kDa (FIG. 7A). Homogeneity of the purified bacteriocin was confirmed by staining with silver nitrate, where a single band was observed (not shown) as for the case of Coomassie Blue staining (FIG. 7A). This band showed an antimicrobial activity against Lact. sakei (FIG. 7B, lane 5).

In the previously reported method by Uteng et al. (2002), a yield of 85% was obtained after the cation-exchange chromatography with an increase of 300-fold of specific activity. In the present case, only the fractions harboring the highest antimicrobial activity after the cation-exchange chromatography were pooled. This fraction selection increased the bacteriocin purity by 725 fold. After the second and last step, we obtained a purification factor of 5 725 fold. This improved method could thus be used to obtain bacteriocin faster, economically and with a higher purity level.

### Amino Acid Sequencing and Mass Determination of MM33 Bacteriocin Tryptic Digestion

10 μl of a solution of pediocin (217 μmol μl⁻¹) was diluted with 56 μl 25 mmol l⁻¹ NH₄HCO₃ then 10 μl of DTT (45 mmol l⁻¹) was added and the solution was incubated at 60° C. for 30 minutes. The solution was allowed to cool at room temperature and was subsequently treated with 10 μl iodoacetamide (100 mmol l⁻¹) and then incubated in the dark at room temperature for 30 minutes. 14 μl of 0.1 μg μl⁻¹ of trypsin was added and the solution was incubated at 37° C. for 4 h. The solution was then quenched with 100 μl of 2% acetonitrile diluted in water and containing 0.1% trifluoroacetic acid.

Amino acid sequencing and mass determination were done by High-Performance Liquid Chromatography combined with Electrospray Ionization Mass Spectrometry. Liquid chromatography (LC) analyses were performed with a nanoLC system, type Agilent 1100 Series (Agilent Technologies Inc., Palo Alto, Calif., USA). A gradient of solvent A (5% acetonitrile in water with 0.1% formic acid) and solvent B (90% acetonitrile in water with 0.1% formic acid) at a flow rate of 0.4 ml min⁻¹ was used. The gradient was as follow: a wash step of 5 minutes with 87% of solvent A was followed with a gradient of 9 minutes to bring the solvent A to 82%. Then, solvent A was reduced to 50% in 10 minutes and to 0% in 3 minutes. Finally, a clean-up step was done during 5 minutes with 100% solvent B. The column switching system consisted of a trap column (ZORBAX 300 SB-C18 reversed phase, 5×0.3 mm, 5 μm particles size (Agilent Technologies Inc.) and an analytical column (ZORBAX 300 SB-C18 reversed phase, 150 mm×75 μm, 3.5 μm particle size (Agilent Technologies Inc.). All mass spectra were recorded on a Linear Ion Trap Quadrupole LC/MS/MS Mass Spectrometer (AB Applied Biosystems, MDS SCIEX Instruments, CA, USA) equipped with a nano-electrospray ionization source. The accumulation of MS-MS data was performed with the Analyst software, version 1.4 (AB Applied Biosystems). Mascot Distiller (Matrix Science, London, UK) was used to create peak lists from MS and MS-MS raw data. Mascot Server (Matrix Science) was used for database searching.

The intact protein was analyzed using this LC-MS system. The amino acid sequence of the bacteriocin produced by P. acidilactici MM33 was predicted by the Mascot software package from LC-MS data. It comprised 44 amino acid residues and the calculated mass of the pediocin was 4 625 Da. The solution which was analyzed contained oxidized and non-oxidized forms. The experimental mass obtained was 4 626 Da for the non-oxidized form and 4 643 Da for the oxidized form (data not shown). The exact amino acid sequence of the bacteriocin produced by P. acidilactici MM33 was as follows:

(SEQ ID NO.: 1)
KYYGNGVTCGKHSCSVDWGKATTCIINNGAMAWATGGHQGNHKC

### MM33 Bacteriocin Gene Amplification

To demonstrate that human P. acidilactici MM33 strain harbors the pediocin PA-1/AcH gene, PCR analysis was performed. The PCR amplification was performed in a 50 μl reaction volume containing 5 μl of 10×PCR reaction buffer plus MgSO₄ (100 mmol l⁻¹ Tris-HCl, pH 8.85, 250 mmol l⁻¹ KCl, 50 mmol l⁻¹ (NH₄)₂SO₄; Roche Canada, Laval, QC, Canada), 2 μl of 25 mmol l⁻¹ MgSO₄, 0.05% Tween-20, 4% propionamide, 0.6 μmol l⁻¹ of 3′ and 5′ end primers, 800 μmol l⁻¹ of deoxynucleotide triphosphate (GE Healthcare, Baie d'Urfé, QC, Canada) and 2.5 U of Pwo DNA polymerase (Roche Canada). About one hundred single colonies were picked with a sterile toothpick from the surface of a MRS agar plate and mixed in the PCR solution. A DNA thermo cycler (Biometra, Montreal Biotech, Dorval, QC, Canada) was used to provide the temperature cycles as follow: 94° C. for 3 minutes, then 30 cycles at 94° C. for 45 s, 50° C. for 45 s and 72° C. for 1 minute were done with the final elongation step at 72° C. for 7 minutes. The primers were designed from pediocin PA-1/AcH structural gene, which were complementary to by 1076 to 1100 (primer 1) and 1238 to 1264 (primer 2). The restriction sites EcoRI and KpnI were added at the 5′ end, of primer 1 and primer 2 respectively, for cloning purpose.

Pediocin 5′
(SEQ ID NO.: 2)
3′ AAAGAATTCATGAAAAAAATTGAAAAATTAACTG 3′
Pediocin 3′
(SEQ ID NO.: 3)
5′ AAAGGTACCCTAGCATTTATGATTACCTTGATGTCC 3′

The primers were used to amplify the potential pediocin gene from total DNA of P. acidilactici MM33 or Lactococcus lactis ATCC 11454 colonies. The amplified PCR products were visualized and purified from a 2% agarose gel using a Qiaquick gel extraction kit (Qiagen, Mississauga, ON, Canada) and the nucleotide sequences were determined by Génome Québec (Montréal, QC, Canada). A single 188-bp fragment was amplified from DNA of P. acidilactici MM33 while no fragment was amplified from DNA neither of P. acidilactici MM33A (described below) nor of L. lactis ATCC 11454 (data not shown). The PCR product DNA was then sequenced and compared to published database. Results indicated 100% homology with pedA gene of P. acidilactici PAC1.0 (data not shown).

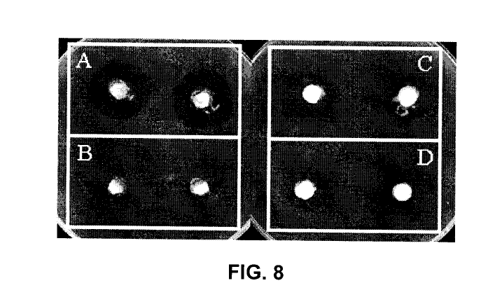

Plasmid curing was performed to determine if the gene encoding the production of pediocin by P. acidilactici MM33 was plasmid linked as reported for other pediocin-like bacteriocins. Tubes of 4.9 ml of MRS medium containing 2.5 μml⁻¹ of novobiocin (Sigma) were inoculated with 50 μl P. acidilactici MM33 and incubated for 24 h at 37° C. Novobiocin was used as a curing agent since it is known that small concentration of this antibiotic prevent plasmid replication via DNA gyrase antagonism (Hooper et al. 1984). Inoculums from each tube were then transferred to fresh MRS-novobiocin medium during the subsequent five days. At the end of the treatment period, ten-fold serial dilutions of each tubes were performed in peptone water (0.1%, w/v, Difco) and appropriate dilutions were spread on MRS agar plates and incubated in an anaerobic jar system (BBL GasPak system, Becton Dickinson and Co., Sparks, Md., USA) at 37° C. After 48 h incubation, 100 bacterial colonies were randomly selected and transferred to MRS agar plates and incubated under anaerobic conditions at 37° C. These isolates were then tested for bacteriocin production using the well-diffusion assay. Proteases were added as a control to confirm the presence of a bacteriocin. A non-bacteriocin-producing colony (ped⁻) was isolated and named P. acidilactici MM33A. FIG. 8A show antimicrobial activity of the supernatant of MM33 while FIG. 8C show that the supernatant of MM33A did not exert an antimicrobial activity. Controls with protease to confirm the protein nature of this antimicrobial activity if shown in FIGS. 8B and 8D.

Plasmid DNA was purified by growing colonies of P. acidilactici MM33 and MM33A overnight in 10 ml of MRS broth at 37° C. Inoculums from these cultures were subsequently grown in 10 ml of fresh MRS overnight. Thereafter, cells from a sample of 1.5 ml were harvested by centrifugation at 1500×g for 20 minutes at 4° C. and the plasmid DNA was isolated using the method of Duan et al. (1999). DNA was stored at −20° C. until it was examined by a 0.7% agarose gel electrophoresis. The plasmid DNA was analyzed by agarose gel electrophoresis. Results show that the novobiocin-treated P. acidilactici MM33A strain lost its plasmid DNA as compared with the plasmid DNA purified from bacteriocin-producing P. acidilactici MM33 strain (data not shown). The pediocin gene homology combined with the loss of bacteriocin activity of P. acidilactici MM33A ped mutant, suggests the plasmid linkage of the pediocin producing gene.

### Example 6

### Inhibitory Activity of Purified Pediocin on L. Monocytogenes

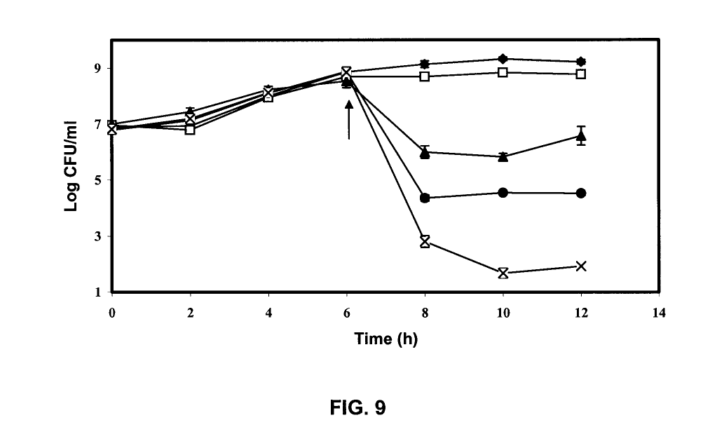

Different amounts of purified bacteriocin (0 to 800 AU/ml) were added to cultures of L. monocytogenes HPB 2812 serotype ½a in early exponential phase (grown for 6 h) and incubated at 37° C. Bacterial growth was monitored by measuring the bacterial population in a 1 ml sample taken every 2 h of incubation. These samples were serially diluted ten-fold in peptone water (0.1% w/v), pour-plated in BHI agar and then incubated at 37° C. for 24 h. Colonies were counted using Darkfield Quebec Colony Counter (American Optical, Scientific instrument division, Keene, Ohio, USA). Results show that viable counts decreased from 9.14 to 8.7, 6.0, 4.36 and 2.81 log CFU ml⁻¹, two hours after the addition of 100, 200, 400 and 800 AU ml⁻¹ of pediocin. The bactericidal activity of pediocin produced by P. acidilactici MM33 against L. monocytogenes cells caused a decrease of more than 99.9% in viable CFU ml⁻¹ when at least 200 AU ml⁻¹ of pediocin were added into the culture medium (FIG. 9).

### Example 7

### Characterization MM19 Bacteriocin

PCR amplification was performed to characterize the MM19 bacteriocin. The primers were designed from nisin and pediocin PA-1/AcH structural genes, which were complementary to regions 17 bp upstream (primer 1) and 2 bp downstream (primer 2) of the coding region for nisin and pediocin.

Nisin primers:

| | |
|---|---|
| 5′ CCGGAATTCATAAGGAGGCACTCAAAATG 3′ | (SEQ ID NO.: 4) |
| 3′ CGGGGTACCTACTATCCTTTGATTTGGTT 5′ | (SEQ ID NO.: 5) |

The amplified PCR products were visualized and purified from a 2% agarose gel using a Qiaquick gel extraction kit (Qiagen, Mississauga, ON, Canada) and the nucleotide sequences were determined by Génome Québec (Montréal, QC, Canada). The 227 bp fragment amplified from the genomic DNA of Lc. lactis MM19 revealed 100% homology to that of nisin Z. The sequence obtained was as follows (underlined is the start of nisin structural gene):

(SEQ ID NO.: 6)
TNC NAA GAT TTT AAC TTG GAT TTG GTA TCT GTT TCG
AAG AAA GAT TCA GGT GCA TCA CCA CGC ATT ACA AGT
ATT TCG CTA TGT ACA CCC GGT TGT AAA ACA GGA GCT
CTG ATG GGT TGT AAC ATG AAA ACA GCA ACT TGT AAT
TGT AGT ATT CAC GTA AGC AAA TAA CCA AAT CAA AGG
ATA GTA GGT ACC CCG AGA NA

### Example 8

### Influence of Lab on Fecal Microbiota

### Animals

Six- to eight-week-old female C57BL/6 mice (Charles River Laboratories, St-Constant, QC, Canada) were used for the evaluation of the fecal microbiota modulation experiment. Mice were housed between 3 and 5 per plastic cages and kept under pathogen-free conditions with free access to commercial diet (Lab diet 5001, Ren's Feed & Supplies, Oakville, ON, Canada) and water. Cages and bedding were changed every two days. This work was approved and supervised by the INRS-Institut Armand-Frappier Animal Care Committee.

Healthy mice received a daily dose of about 10⁹ viable bacteria (Lc. lactis subsp. lactis MM19, P. acidilactici MM33 or P. acidilactici MM33A) in 100 ml of PBS by intragastric route using a stainless steel feeding needle and a 1 ml syringe. A group of mice received PBS as a negative control. Mice were weighed at days 1, 9, 18 and 27 (day 27 representing 9 days after the end of the feeding treatment) and any sign of physiological or behavioral perturbation were noticed during the experiment. Stool samples were collected before the administration of PBS or LAB (day 1), as well as 9 and 18 days after the beginning of the feeding procedures. Final analysis was done 9 days after the end of the treatment (27 post feeding day). The stools were collected directly after defecation in a pre-weighted 2-ml sterile plastic tube. These tubes were kept on ice until microbial analysis. Fecal populations of total culturable LAB, Lactobacillus spp., anaerobes, Enterobacteriaceae, Staphylococcus spp. and Enterococcus spp. were analyzed on selective media. The experiment was repeated twice using a total of ten mice per experimental group.

### Quantification of Stool Organisms

The tubes containing fresh stool samples, collected from individual mice, were weighted, so that the weight of the feces could be deduced. Then, all the feces were diluted in 1000 ml of sterile saline, homogenized with a pestle, and serially diluted 10-fold in 0.1% peptone water. Finally, 100 ml of each dilution were inoculated on the following media: MRS agar for detection of total lactic acid bacteria (LAB), Rogosa SL agar for selective detection of Lactobacillus spp., Reinforced Clostridium Medium (RCM) for quantification of total anaerobic and mesophilic bacteria, Baird-Parker agar (BPA) for selective detection of Staphylococcus spp., MacConkey agar for selective enumeration of Enterobacteriaceae, Enterococcosel agar for selective quantification of total Enterococcus spp. and finally Enterococcosel agar+20 μg/ml of vancomycin (Sigma) for detection and enumeration of VRE. A volume of 100 μl of the undiluted sample was also plated. MRS, Rogosa and RCM plates were incubated in anaerobic jars at 37° C. for 72 h while BPA, MacConkey and both Enterococcosel agar plates were incubated under aerobic conditions at 37° C. for 48 h. When negative results were obtained for Enterococcosel agar+20 μg/ml of vancomycin after 48 h of incubation, the plates were allowed to incubate for another 24 h. For statistical purposes, a value in CFU per gram that was based upon the weight of individual specimens was assigned for faeces without microorganisms.

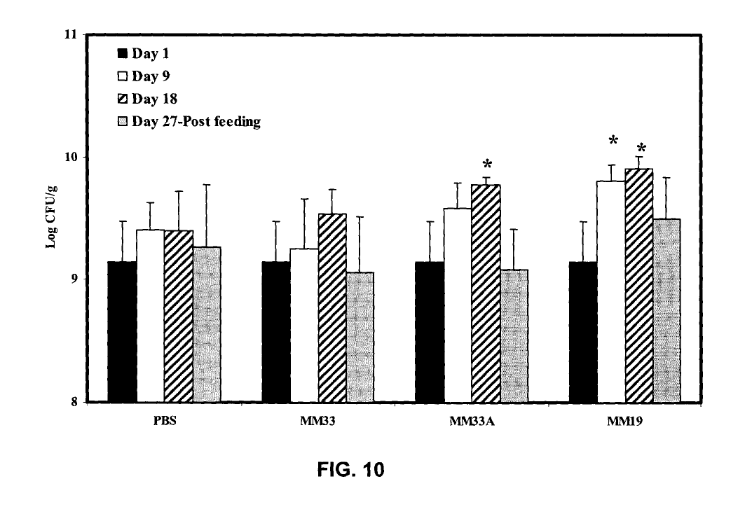

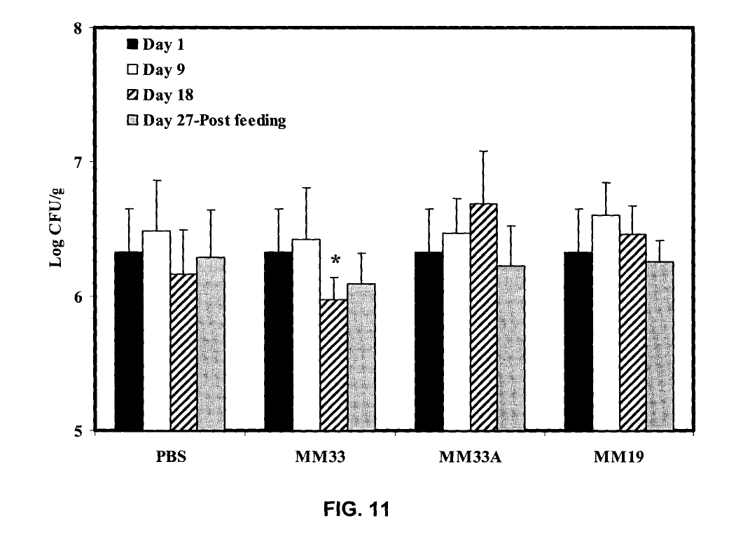

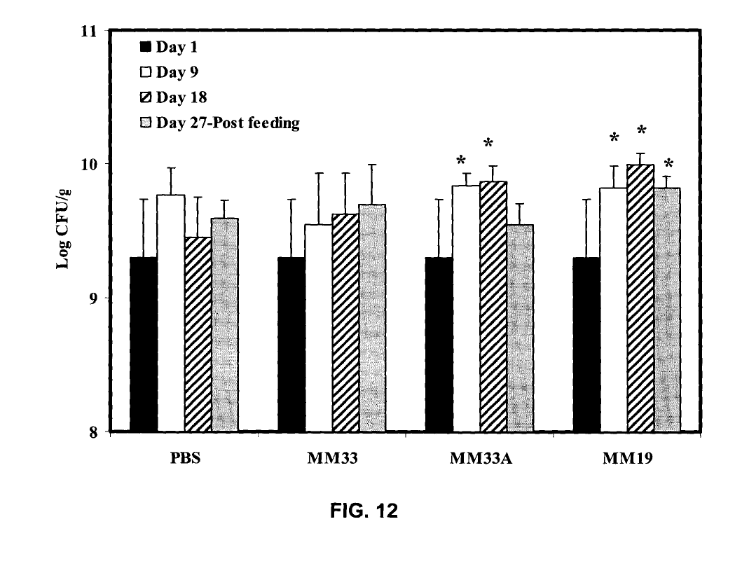

The influence of bacteriocin-producing LAB ingestion on the fecal microbial populations of healthy C57BI/6 is shown in FIGS. 10 to 12. The LAB counts in feces of mice fed with L. lactis subsp. lactis MM19 is higher (P≦0.05) after 9 and 18 days of feeding. However, after feeding interruption, the level of LAB was similar to its initial count. Ingestion of P. acidilactici MM33A, the non pediocin-producing mutant, led to an increase (P≦0.05) of LAB after 18 days while the pediocin-producing strain did not quantitatively influence the LAB population (FIG. 10). P. acidilactici MM33 reduced significantly the Enterobacteriaceae population in mice feces after 18 days of feeding (FIG. 11). This strain modified the microbial balance in the gut, leading to the reduction of the Enterobacteriaceae. Results shown in FIG. 12 indicate that MM19 feeding increased (P≦0.05) the culturable anaerobic population significantly after 9 and 18 days. This increase was maintained after feeding ended. The non pediocin-producing strain increased the anaerobes as long as the feeding lasted but after feeding ended, the level of anaerobes was similar to its initial count. Ingestion of bacteriocin-producing bacteria is well tolerated by C57BI/6 mice over the course of a three week-feeding trial and can alter quantitatively the balance of colonic bacterial populations.

### Example 9

### VRE Intestinal Colonization

A VRE intestinal colonization (infection) model was adapted from Donskey et al. 25-30 g CF-1 female mice (Charles River) were used. Daily subcutaneous administration of clindamycin (1.4 mg/d) for five days was used to disrupt the intestinal microbiota to induce VRE infection. Three days after the end of clindamycin administration, gastric inoculation of 250 μl of an overnight culture of VRE in BHI broth was used to infect the mice. Approximately 10⁸ viable VRE was administered to the mice. Since the beginning of the antibiotic therapy, all groups of LAB-treated mice received once daily 100 ml of 10¹⁰ CFU/ml of Lc. lactis subsp. lactis MM19, P. acidilactici MM33 or P. acidilactici MM33A previously washed twice in sterile PBS. The mice received the LAB until the eighth day after the infection. Bacitracin (Sigma) antibiotic was administrated orally once daily during three days following the mice infection with VRE as 600 units/d diluted in 100 ml of sterile PBS. This treatment was then discontinued and replaced by PBS. Stool samples were collected before the antibiotic administration and 1, 3, 6, 8 and 12 days after the VRE infection. Fecal concentrations of total culturable VRE were analyzed using selective media. This experiment was repeated twice using a total of eight mice per experimental group.

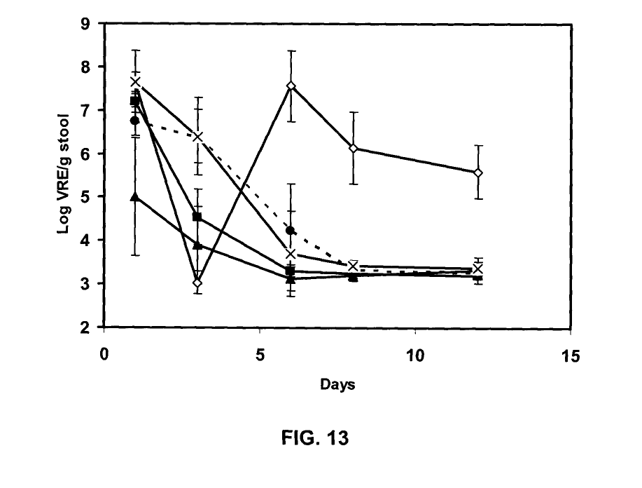

Results presented in FIG. 13 show that the VRE densities one day post infection was lower by 1.73 log₁₀ CFU/g (P≦0.05) for the group of L. lactis subsp. lactis MM19-fed mice as compared to the PBS-fed group. Moreover, in comparison with PBS controls, Lc. lactis MM19 and P. acidilactici MM33-treated mice have significantly lower VRE densities 3 days after the infection (P≦0.05). The VRE population was reduced by 2.50 and 1.85 log₁₀ CFU/g, respectively. Six days after the infection, undetectable level of VRE was measured in L. lactis subsp. lactis MM19 and P. acidilactici MM33 treated mice. VRE densities in mice feces of the group fed with the non pediocin-producing strain were similar to the level measured for controls for the duration of the experiment. Mice treated with bacitracin during three days after the infection had an undetectable level of VRE at day 3. However, the VRE population reappeared when the bacitracin treatment was discontinued as previously reported. It is interesting to note that when the oral administration of the mice with LAB was discontinued (8 days after the infection), no recurrence of the VRE was observed 4 days later.

Results presented in TABLE 8 show that one day after the VRE infection, all mice were colonized (infected) by the pathogen with the exception that 83% of mice fed with L. lactis subsp. lactis MM19 showed detectable level of VRE. A similar trend was observed 3 days after the infection with 71% of colonization for the Lc. lactis MM19-treated mice while all the other mice showed detectable level of VRE. However, six days following the infection, no mice that had received bacteriocin-producing strains were colonized with the pathogen while 60% and 50% of the PBS and P. acidilactici MM33A-treated group were colonized, respectively. The bacteriocin-producing strains are therefore able to reduce the densities of VRE population following an infection induced by clindamycin.

TABLE 8
CF-1 MICE COLONIZED (%) WITH DETECTABLE LEVEL OF VRE

| | Mice colonized with VRE^{a} (%) | | | | | |
|---|---|---|---|---|---|---|
| | Days after VRE infection | | | | | |
| | 0 | 1 | 3 | 6 | 8 | 12 |
| PBS | 0 | 100 | 100 | 60 | 0 | 0 |
| Bacitracin | 0 | 100 | 0 | 100 | 100 | 100 |
| Lc. lactis MM19 | 0 | 83 | 71 | 0 | 0 | 0 |
| P. acidilactici MM33 | 0 | 100 | 100 | 0 | 0 | 0 |
| P. acidilactici MM33A | 0 | 100 | 100 | 50 | 0 | 0 |

^{a}A total of eight mice were used in two independent experiments for each group.

The data presented above show that the MM19 and MM33 LAB strains producing bacteriocins (nisin and pediocin) are capable of modulating the intestinal microbiota of healthy mice and to impact (reduce) the intestinal colonization of VRE-infected mice.

Although the present invention has been described by way of exemplary embodiments, it should be understood by those skilled in the art that the foregoing and various other changes, omissions and additions may be made therein and thereto, without departing from the spirit and scope of the present invention as defined in the appended claims.

### REFERENCES

Altschul et al. 1990 J Mol Bio 215 403-410
Carolissen-Mackay et al. 1997 Int J Food Microbiol 34, 1-16
Cutler P, “Protein Purification Protocols” Humana Press, 2004, 484 pages
Delgado, S., et al. 2005. Current Microbiology, 50: 202-207
Donskey et al. 2001 Lett Appl Microbiol 33, 84-88
Drider et al. 2006 Microbiol Mol Biol Rev 70, 564-582
Duan et al. 1999 Biotechnol Tech 13, 519-521
Hooper et al. 1984 Antimicrob Agents Chemother 25, 586-590
Riley and Gordon, 1999 Trends Microbiol 7 129-133
Schillinger and Lucke 1989 Appl Environ Microbiol 55, 1901-1906
Toure et al. 2003 J Appl Microbiol 95, 1058-1069
Uteng et al. 2002 Appl Environ Microbiol 68, 952-956

1. An isolated lactic acid bacteria having the identifying characteristics of a strain selected from the group consisting of Lactococcus lactis subsp. lactis MM19 accession number NML-080508-01 and Pediococcus acidilactici accession number NML-080508-02.

2. A culture of the isolated lactic acid bacteria of claim 1.

3. A cell-free culture supernatant of the culture of claim 2.

4. The cell-free culture supernatant of claim 3 wherein the supernatant is concentrated.

5. The cell-free culture supernatant of any one of claim 3 or 4 wherein the supernatant is neutralized.

6. An isolated bacteriocin produced by the lactic acid bacteria of claim 1.

7. The isolated bacteriocin of claim 6 wherein the bacteriocin is selected from the group consisting of nisin and pediocin.

8. A composition comprising the isolated lactic acid bacteria of claim 1, the culture of claim 2, the cell-free culture supernatant of claim 3, the isolated bacteriocin of claim 6 or combination thereof and a carrier.

9. A food product comprising the isolated lactic acid bacteria of claim 1, the culture of claim 2, the cell-free culture supernatant of claim 3, the isolated bacteriocin of claim 6 or combination thereof.

10. A method for inhibiting microbial growth comprising the step of contacting a microbe with the isolated lactic acid bacteria of claim 1, the culture of claim 2, the cell-free culture supernatant of claim 3, the isolated bacteriocin of claim 6, the composition of claim 8, the food product of claim 9 or combination thereof.

11. A method for preventing or treating a microbial infection in a mammal in need thereof comprising administering an effective amount of the composition of claim 8 or the food product of claim 9 to the mammal.

12. The method of claim 11 wherein the administration is oral administration

13. A method for preventing or reducing the level of microbial colonization in a food product comprising contacting the food product with an effective amount of the composition of claim 8.

14. The method of any one of claims 10 to 13 wherein the microbes responsible for the microbial growth, microbial infection or microbial colonization are selected from the group consisting of gram-negative and gram-positive bacteria.

15. The method of claim 14 wherein the microbes are gram-positive bacteria.

16. The method of any one of claims 10 to 15 wherein the microbes are antibiotic resistant bacteria.

17. The method of any one of claims 10 to 16 wherein the microbes are pathogenic bacteria.

18. The method of any one of claim 14 or 15 wherein the genus of gram positive bacteria is selected from the group consisting of Enterococcus, Kocuria, Lactobacillus, Listeria, Pediococcus and Staphylococcus.

19. The method of claim 16 wherein the antibiotic resistant bacteria are vancomycin resistant enterococcus.

20. A method for modulating the gut flora in a mammal in need thereof comprising administering an effective amount of the composition of claim 8 or the food product of claim 9 to the mammal.

21. A kit comprising at least one container containing the isolated lactic acid bacteria of claim 1, the culture of claim 2, the cell-free culture supernatant of claim 3, the isolated bacteriocin of claim 6, the composition of claim 8 or the food product of claim 9.
